(19) United States
(12) Patent Application Publication
(10) Pub. No.: US 2023/0226358 A1
(43) Pub. Date: Jul. 20, 2023

(54) Neural Stimulator Impedance Control and Matching

(71) Applicant: URO MEDICAL CORPORATION, Boca Raton, FL (US)

(72) Inventors: Richard Lebaron, Surprise, AZ (US); Patrick Larson, Boca Raton, FL (US)

(21) Appl. No.: 18/001,215

(22) PCT Filed: Jun. 8, 2021
(86) PCT No.: PCT/US21/36416

§ 371 Date: Dec. 8, 2022

Related U.S. Application Data
(60) Provisional application No. 63/036,661, filed on Jun. 9, 2020.

(51) Int. Cl. A61N 1/36

(52) U.S. Cl. CPC A61N 1/36139; A61N 1/3614

(57) ABSTRACT

A method, system, and apparatus for temporarily modifying an impedance of a neural stimulator. The apparatus includes an antenna comprising a first pole and a second pole, a switching circuit configured to output switched signals, a rectifier configured to receive switched signals from the switching circuit, a plurality of electrodes, and a controller, wherein the switching circuit, based on the control signal, modifies one or more of a first pole signal or a second pole signal. The impedance may be modified via one or more switches in a switching circuit of the neural stimulator. The impedance change may be sensed by an external circuit. Also, an electrode-tissue impedance of the neural stimulator may be determined and an impedance of an external circuit modified based on the electrode-tissue impedance of the neural stimulator.

### TECHNICAL FIELD

This disclosure relates to systems and methods for operation of an implantable neural stimulator that can modulate excitable tissue mediums in the body.

### BACKGROUND

Modulation of excitable tissue in the body by electrical stimulation has become an important type of therapy for patients with chronic disabling conditions, including pain, movement initiation and control impairments, autonomic nervous system deficiencies, overactive bladder, inflammation, involuntary movement disorder, vascular insufficiency, heart arrhythmias and various other modalities involving the nervous system. A variety of therapeutic intra-body electrical stimulation techniques may be used to provide therapeutic relief for these conditions. For instance, implantable devices may be used to deliver signals to excitable tissue, record vital signs, perform pacing or defibrillation operations, record action potential activity from targeted tissue, control drug release from time-release capsules or drug pump units, or interface with the auditory system to assist with hearing.

### SUMMARY

In one or more aspects, a method, system, and apparatus for temporarily modifying a radio frequency (RF) signal of a neural stimulator adjusted for tissue impedance are disclosed. The electrode tissue impedance of the neural stimulator may be determined based on its observed effect upon the RF impedance of the neural stimulator itself.

The RF impedance may be modified via one or more switches in a switching circuit enclosed within the neural stimulator. The RF impedance change may be sensed wirelessly by an external circuit. Also, in other aspects, the electrode tissue impedance of the neural stimulator may be determined and an impedance of an external circuit modified based on the electrode-tissue impedance of the neural stimulator.

In one or more aspects of the disclosure, an apparatus may comprise: an antenna comprising a first pole and a second pole, a switching circuit configured to receive a first pole signal from the first pole and configured to receive a second pole signal from the second pole, wherein the switching circuit is configured to output switched signals, a rectifier configured to receive switched signals from the switching circuit, a plurality of electrodes, a controller configured to receive power from the rectifier, configured to selectively power the electrodes, and configured to output a control signal to the switching circuit, wherein the switching circuit, based on the control signal, modifies one or more of the first pole signal or the second pole signal. In some aspects, the switching circuit may comprise a first switch comprising an input configured to receive the first pole signal and configured to output the first pole signal as one of the switched signals, wherein the first switch, based on the control signal, prevents the first pole signal from being output as one of the switched signals. Further, the controller may be configured to output a second control signal, and wherein the switching circuit may further comprise a second switch comprising an input configured to receive the second pole signal and configured to output the second pole signal as another of the switched signals, wherein the second switch, based on the second control signal, prevents the second pole signal from being output as the another of the switched signals. Further, the switching circuit may further comprise a first switch comprising an input configured to receive the first pole signal and configured to output the first pole signal as one of the switched signals; a second switch comprising an input configured to receive the second pole signal and configured to output the second pole signal as another of the switched signals; and wherein the first switch, based on the control signal, prevents the first pole signal from being output as one of the switched signals, and wherein the second switch, based on the control signal, prevents the first pole signal from being output as one of the switched signals. Alternately or additionally, the switching circuit may comprise a first switch comprising a first terminal connected to the first pole and a second terminal connected to the second pole, wherein the first switch, based on the control signal, shorts the first pole and the second pole. Alternately or additionally, the switching circuit may comprise a first switch comprising a first terminal and a second terminal, wherein the first terminal is connected to the first pole; and a load connected between the second terminal and the second pole, wherein the first switch, based on the control signal, connects the load to the first pole. In some aspects of the disclosure, the load may comprise a diode (including but not limited to a conventional diode (e.g., permitting current to flow in only one direction), a light emitting diode, or another type of diode or even a plurality of diodes). In some aspects, the apparatus may further comprise a second switch comprising a third terminal connected to the first pole and a fourth terminal connected to the second pole, wherein the second switch, based on the control signal, shorts the first pole and the second pole. In some aspects of the disclosure, the switching circuit may modify an RF impedance of the apparatus.

In some aspects of the disclosure, a method may comprise receiving, at an antenna, a radio frequency signal, wherein the antenna comprises a first pole and second pole, and wherein the antenna comprises a first radio frequency impedance, receiving, via an input of a switching circuit and from the antenna, the radio frequency signal, selectively outputting, via an output of the switching circuit and based on a control signal from a controller, a switched radio frequency signal, receiving, at a rectifier, the switched radio frequency signal, wherein the selectively outputting interrupts, based on the control signal, a conduction path between the input of the switching circuit and the output of the switching circuit, wherein the controlling operation modifies the antenna to comprise a second radio frequency impedance, and wherein the second radio frequency impedance is different from the first radio frequency impedance. The selectively outputting may further comprise receiving, via a control signal line, the control signal; modifying, based on the control signal, a conduction between a first terminal of a switch of the switching circuit and a second terminal of the switch of the switching circuit, wherein the modifying the conduction of the switch creates on open circuit between the first terminal and the second terminal. The selectively outputting may further comprise modifying, based on the control signal, a conduction between a third terminal of a second switch of the switching circuit and a fourth terminal of the second switch of the switching circuit, wherein the modifying the conduction of the switch creates on open circuit between the third terminal and the fourth terminal. Alternatively or additionally, the selectively outputting may further comprise modifying, based on a second control signal, a conduction between a third terminal of a second switch of the switching circuit and a fourth terminal of the second switch of the switching circuit, wherein the modifying the conduction of the switch creates on open circuit between the third terminal and the fourth terminal. Alternatively or additionally, the selectively outputting may further comprise modifying, based on the control signal, a conduction between a first terminal of a first switch of the switching circuit and a second terminal of the second switch of the switching circuit, wherein the first terminal is connected to the first pole of the antenna, wherein the second terminal is connected to the second pole of the antenna, and wherein modifying the conduction comprises creating a short circuit between the first pole and the second pole of the antenna.

In further aspects of the disclosure, a method may comprise of determining a first impedance at which a neural stimulator begins to respond to an input radio frequency signal from an external antenna and the corresponding normalized impedance radio frequency signal rises above a background noise floor; determining a scaling factor dependent on relative signal strengths of the measurement test setup; determining, based on the electrical load impedance, the first impedance value, and the scaling factor, an estimated electrode-tissue impedance of the neural stimulator; and outputting the estimated electrode-tissue impedance of the neural stimulator. For example, the output may include modifying, based on the estimated electrode-tissue impedance of the neural stimulator, an impedance of the external antenna. In one or more aspects the determining the estimated electrode-tissue impedance of the neural stimulator may be based on a model of normalized impedance RF signal, where:

Model=sqrt((Z−A)/B)

wherein Z is the estimated electrode-tissue impedance for the neural stimulator, wherein A is the impedance value at which the normalized impedance RF signal rises above a background noise floor, and wherein B is a scaling factor dependent on relative signal strengths of the measurement test setup. In further aspects, modifying the impedance of the external antenna may comprise of adjusting an impedance matching circuit of the external antenna. Additionally or alternatively, modifying the impedance may further comprise adjusting an impedance matching circuit of the neural stimulator.

In a further aspect, a method may comprise receiving a radio frequency signal at an antenna of a neural stimulator; determining a voltage across a rectifier of the neural stimulator; determining a time, based on phases of the radio frequency signal, of a voltage drop across the rectifier; determining, based on the voltage across the rectifier and the time, a resistance-capacitance time constant of the neural stimulator; determining, based on the resistance-capacitance time constant, an electrode-tissue impedance of the neural stimulator; and outputting the estimated electrode-tissue impedance of the neural stimulator. For example, the output may include modifying, based on the electrode-tissue impedance of the neural stimulator, an impedance of the external antenna. Alternatively or additionally, modifying the impedance of the external antenna may comprise of adjusting an impedance matching circuit of the external antenna.

These and other aspects are described herein.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1 shows a high-level diagram of an example of a wireless stimulation system.

FIG. 2A shows an example programming controller of the wireless stimulation system of FIG. 1. FIG. 2B shows an example transmitter of the wireless stimulation system of FIG. 1. FIG. 2C shows an example neural stimulator of the wireless stimulation system of FIG. 1.

FIG. 3A is an example of a transmitter for wireless power transfer to a neural stimulator dipole antenna. FIG. 3B is another example of the transmitter with a directional coupler and power detector. FIG. 3C is a graph of sensor VSWR and backscatter signaling. FIG. 3D is a graph showing path loss based on antenna alignment.

FIGS. 4A-4D are examples of normalized reflection versus normalized power under various loading conditions.

FIG. 5 is an example of a flow chart.

FIGS. 6A-6I show various examples from simulation computations for RL and VSWR-based neural stimulator location detection.

FIG. 7 is an example of backscatter modulation.

FIG. 8 is an example of stimulation current compared to RF power.

FIG. 9 is an example of a detection signal compared to a frequency shift.

FIG. 10 is the example a signal-to-noise ratio.

FIGS. 11-12 are examples of a detection signal compared to a phase shift.

FIG. 13 is an example of a circulator in a transmitter.

FIG. 14 is an example of a switch controlling the impedance of a neural stimulator.

FIGS. 15 and 16 are examples of the detection of backscattering of a stimulation signal.

FIG. 17 is an example of an impedance signal compared to impedance.

FIG. 18 is an example of a transmitter with an impedance matching circuit.

FIG. 19 is an example of an external antenna with an impedance matching circuit.

### DETAILED DESCRIPTION

Certain aspects of the disclosure relate to applying a current of a neural stimulator. In some aspects of the disclosure, the neural stimulator may comprise one or more circuits to vary an RF impedance of a neural stimulator antenna. By varying the RF impedance, the neural stimulator may communicate wirelessly with an external controller. Also, certain aspects of the disclosure relate to increasing a signal-to-noise ratio and/or power transfer capability of the external controller to address varying degrees of electrode-tissue impedance that vary over time or vary based on proximity to surrounding tissues. As used herein, the term “electrode-tissue impedance” refers to the impedance at the neural stimulator electrodes. Electrode-tissue impedance may include pure resistance, pure reactance, or a combination of both (resulting in a complex impedance having both real and reactive components).

A wireless stimulation system may include a neural stimulator device with one or more electrodes and one or more conductive antennas (for example, a dipole antenna or a patch antenna or other type of antenna), and internal circuitry for detecting pulse instructions, and rectification of RF electrical signal. The system may further comprise an external controller with an antenna for transmitting radio frequency energy from an external source to the neural stimulator with neither cables nor inductive coupling to provide power. The neural stimulator may be implanted into a patient (e.g., through an incision in a person's skin) or inserted into a patient (e.g., into a person's mouth or nasal cavity or other openings).

In various implementations, the neural stimulator is configured to receive power transmitted wirelessly from the external controller. The neural stimulator may receive power through a wireless coupling with the external controller. For example, an antenna of the neural stimulator may receive RF power through an electrical radiative coupling with an antenna of the external controller. The received RF power may be used to power the neural stimulator to permit the neural stimulator to stimulate nerve bundles without the neural stimulator having a physical connection to an internal battery or without the use of an inductive coil.

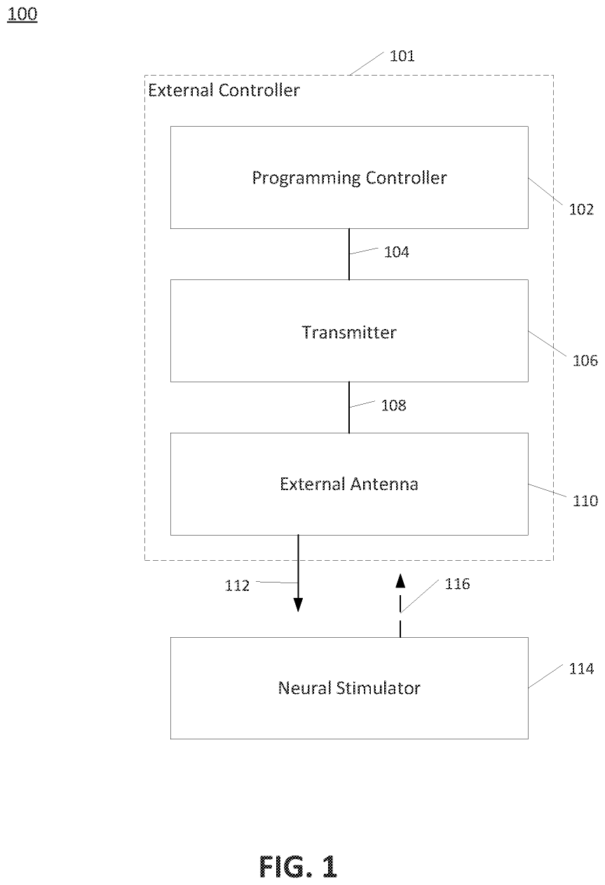

FIG. 1 shows a high-level diagram of an example of a wireless stimulation system. The wireless stimulation system 100 may include four general functional blocks, e.g., a programming controller 102, a transmitter 106, an external antenna 110 (for example, a patch antenna, a slot antenna, a dipole antenna, and/or other known antenna types), and a neural stimulator 114. The programming controller 102 may be a computer device, such as a smart phone, running one or more software applications that use a wireless connection 104 of the programming controller 102. The wireless connection may include wireless connection protocols such as Bluetooth®, Wi-Fi, Zigbee, and/or other wireless connection protocols. The software application may enable the user to view the system status of the wireless stimulation system 100 and start diagnostics, change various parameters, increase/decrease the desired stimulus amplitude of the electrical pulses, and adjust feedback sensitivity of the transmitter 106, among other functions.

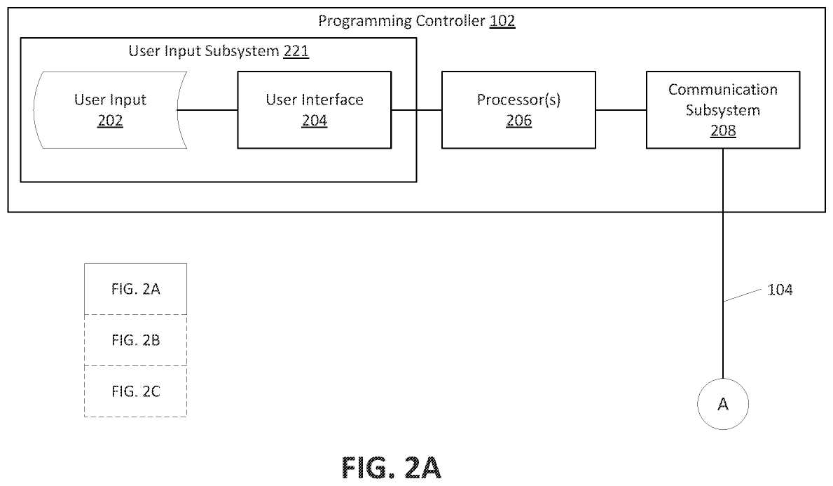

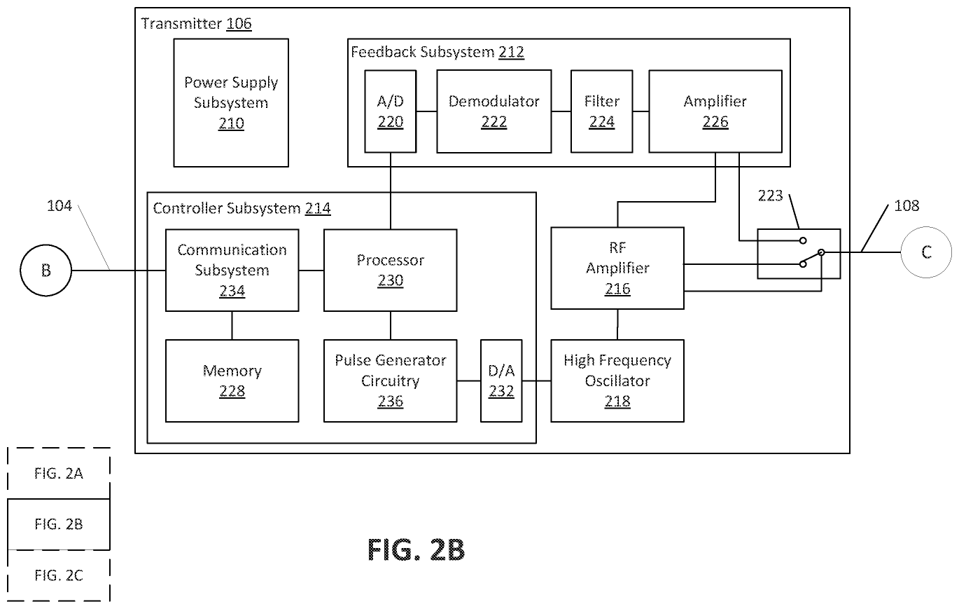

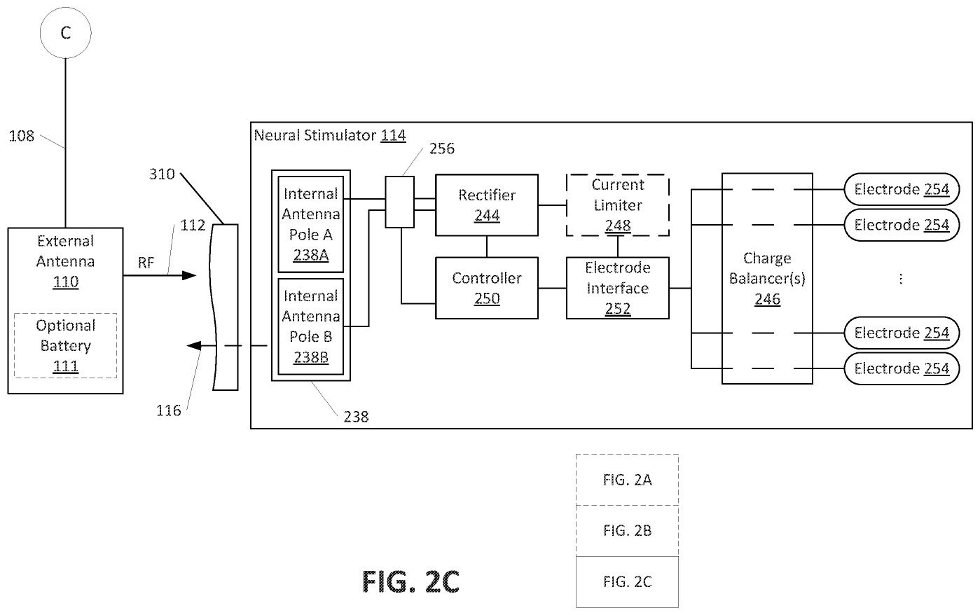

The transmitter 106 may include communication electronics that support the wireless connection 104 and a battery (or other power source) to power the generation of a radio frequency, radiative signal 112. In some implementations, the transmitter 106 may include an external antenna 110. The external antenna 110 may be part of the housing of the transmitter 106 or may be separate from the housing of the transmitter 106. If separate, it may be connected to the transmitter 106 via a wired connection 108 or via a wireless connection (not shown). Further, the external antenna 110 may optionally include its own power source (e.g., including a battery 111 described in FIG. 2C herein).

The external antenna 110 may create an electric field that powers the neural stimulator 114. In addition to providing power to the neural stimulator, the external controller 101 may provide instructions, via external antenna 110, to the neural stimulator 114. The power and/or instructions are represented generally as radiative signal 112. Further, the neural simulator 114 may also communicate with the external controller 101 via a backscatter signal 116. For instance, the external antenna 110 radiates an RF transmission signal (e.g., radiative signal 112) that is modulated and encoded by the transmitter 106. The neural stimulator 114 contains one or more antennas (e.g., a dipole or patch or other antenna design). The radiative signal 112 is received via the one or more antennas. In various examples, the external antenna 110 may be electrically coupled (also referred to as electrical radiative coupling) to the neural stimulator 114 and not inductively coupled. In other words, the coupling is through an electric field rather than a magnetic field.

Through this electrical radiative coupling, the external antenna 110 may provide the radiative signal 112 to the neural stimulator 114. This radiative signal 112 may deliver energy to power the neural stimulator 114 and may contain information encoding instructions regarding stimulus waveforms to be applied, via electrodes of the neural stimulator 114, to surrounding tissues and/or instructions to alter the RF impedance of the neural stimulator 114. In some implementations, a power level of the radiative signal 112 may be used to indicate the amplitude (for example, power, current, or voltage) of the one or more electrical pulses applied by the neural stimulator 114 to the surrounding tissues. Additionally or alternatively, the intensity of the radiative signal 112 may be independent of the intensity of the pulses applied by the neural stimulator 114 to the surrounding tissue.

One or more circuits in the neural stimulator 114 may receive radiative signal 112 and generate, using the energy contained in the radiative signal 112, the pulses suitable for the stimulation of neural tissue.

In some implementations, the external controller 101 may remotely control the stimulus parameters (that is, the parameters of the electrical pulses applied to the neural tissue) and monitor feedback from the neural stimulator 114 based on the backscatter signals 116 received from the neural stimulator 114. A feedback detection algorithm implemented by the transmitter 106 may monitor data sent wirelessly via backscatter signal 116 from the neural stimulator 114, including information about the energy that the neural stimulator 114 is receiving from the external controller 101 and information about the stimulus waveform being delivered to the electrodes. In order to provide an effective therapy for a given medical condition, the wireless stimulation system 100 may be tuned to provide an optimal amount of excitation or inhibition to the nerve fibers by electrical stimulation. A closed-loop feedback control method may be used in which the output signals from the neural stimulator 114 are monitored by external controller 101 and used to determine the appropriate level of neural stimulation for maintaining effective therapy. Additionally or alternatively, an open-loop control method may be used.

FIGS. 2A-2C show examples of the wireless stimulation system of FIG. 1. In FIG. 2A, a programming controller 102 may comprise a user input subsystem 221 and a communication subsystem 208. The user input subsystem 221 may allow various parameter settings to be adjusted (in some cases, in an open-loop fashion or in a closed-loop fashion) by the user in the form of instruction sets. The communication subsystem 208 may transmit these instruction sets (and other information) via the wireless connection 104, such as Bluetooth or Wi-Fi, to the transmitter 106, as well as receive data from the transmitter 106. Additionally or alternatively, the programming controller 102 may be integrated into the housing of the transmitter 106.

For instance, the programming controller 102, which may be used for multiple users, such as a patient's control unit or the clinician programmer unit, may be used to send stimulation parameters for an intended therapy to the transmitter 106, which in turn may encode one or more of these stimulation parameters into the radiative signal 112 transmitted to neural stimulator 114. The stimulation parameters that may be controlled may include pulse amplitude, pulse repetition rate, and pulse width in the ranges shown in Table 1. In this context the term “pulse” refers to the “stimulus phase” of the stimulus waveform output by the neural stimulator 114 that directly produces stimulation of the neural tissue; the parameters of the charge-balancing phase (described below) may similarly be controlled. The patient and/or the clinician may also optionally control overall duration and pattern of therapy.

TABLE 1

| Stimulation Parameter: | Range: | |
|---|---|---|
| Pulse Amplitude | 0 to 25 | mA |
| Pulse Repetition Rate | 0 to 20000 | Hz |
| Pulse Width | 0 to 2 | ms |

The transmitter 106 may be pre-programmed during manufacturing and/or it may be field programmed (e.g., programmed after manufacturing by a clinician and/or user) to encode the stimulation parameters (e.g., parameter-setting attributes) for neural stimulator 114 to meet the specific therapy requirements for each individual patient. Because medical conditions or the tissue properties can change over time, the ability to re-adjust the stimulation parameters may be beneficial to ensure ongoing efficacy of the neural modulation therapy.

The programming controller 102 may be functionally a smart device and associated application. The user input subsystem may comprise a user interface 204 that receives user input 202 and forwards that input to one or more processors 206. The one or more processors 206 are shown as separate from the user input but may include one or more processors as part of the user input subsystem 221 and separate processors for the remaining subsystems of the programming controller 102. For example, the user interface 204 may be a touch screen as part of graphical user interface and/or a display with separate buttons.

FIG. 2B shows an example transmitter of the wireless stimulation system of FIG. 1. The signals sent by transmitter 106 to the neural stimulator 114 may include both power and parameter-setting attributes in regards to a stimulus waveform to be output by electrodes of the neural stimulator 114. The parameter-setting attributes may comprise one or more of parameters relating to pulse amplitude, pulse width, or pulse repetition rate. Additionally or alternatively, the parameter setting attributes may comprise instructions to change an operation mode of the neural stimulator 114. The transmitter 106 may also function as a wireless receiving unit that receives feedback signals from the neural stimulator 114. The transmitter 106 may contain microelectronics or other circuitry to handle the generation of the signals transmitted to the neural stimulator 114 as well as handle feedback signals, such as those from the neural stimulator 114. The transmitter 106 includes various subsystems comprising, in general, a power supply subsystem 210, a controller subsystem 214, and a feedback subsystem 212. The power supply subsystem 210 may comprise a transformer (e.g., AC-to-DC) and a wired connection to an AC signal source, a DC power input (connected to a DC power supply, not shown), and/or an onboard battery configured to store electrical energy for use by the transmitter 106.

The controller subsystem 214 may comprise a communication subsystem 234 that receives a signal via the wireless connection 104 from the programming controller 102 of FIG. 2A. The communication subsystem is communicatively coupled with a memory 228 and one or more processors 230 (for simplicity, shown as a single processor 230). The processor 230 controls the pulse generator circuitry 236 to generate waveforms to be transmitted to the neural stimulator 114.

The controller subsystem 214 may be used by the patient and/or the clinician to control the stimulation parameter settings (for example, by controlling the parameters of the signal sent from transmitter 106 to the neural stimulator 114). These parameter setting attributes may affect, for example, the power, current level, pulse width, pulse repetition rate, or shape of the one or more electrical pulses. The programming of the stimulation parameters may be performed using the programming controller 102, as described above, to set the pulse repetition rate, pulse width, amplitude, and waveform that will be transmitted by radiative signal 112 to the internal antenna 238 (hereinafter referred to as the stimulator antenna 238), typically a dipole antenna (although other types may be used), in the neural stimulator 114. The clinician may have the option of locking and/or hiding certain settings within the programming controller 102, thus limiting the patient's ability to view or adjust certain parameters because adjustment of certain parameters may require detailed medical knowledge of neurophysiology, neuro-anatomy, protocols for neural modulation, and safety limits of electrical stimulation.

The controller subsystem 214 may store received parameters in the memory 228, until the parameters are modified by new data received from the programming controller 102. The processor 206 may use the parameters stored in the local memory to control the pulse generator circuitry 236 to generate a pulse timing waveform that modulates a high frequency oscillator 218 that may generate an RF carrier frequency in the range from 300 MHz to 8 GHz (e.g., between about 700 MHz and 5.8 GHz and, with a tighter range, between about 800 MHz and 1.3 GHz). The controller subsystem 214 may further comprise a digital-to-analog converter (D/A) 232 that converts a digital form of received waveforms to their analog complement. The analog version of the waveforms are conveyed to a high frequency oscillator 218, where the analog waveforms modulate a carrier frequency into a composite signal. The composite signal is conveyed to a radiofrequency (RF) amplifier 216. The radio frequency amplifier 216 amplifies the received composite signal and outputs an amplified composite signal to an RF switch 223. The controller subsystem 214 also controls the operation of the RF switch 223 based on whether radio frequency amplifier 216 is actively transmitting the composite signal or feedback subsystem 212 is waiting for a possible backscatter signal 116 from the neural stimulator 114.

The amplified composite signal may be conducted through an RF switch 223 to the external antenna 110, which converts the amplified composite signal into radiative signal 112. The transmitter 106 may adjust the amplitude of the amplified composite signal as needed. In some implementations, the amplitude of the amplified composite signal may be increased or decreased to compensate for attenuation of radiative signal 112 caused by depths of tissue in the pathway from external antenna 110 to the stimulator antenna 238. In some implementations, the RF signal 112 sent by external antenna 110 may simply be a power transmission signal used by the neural stimulator 114 to generate electric pulses. In some implementations, the RF signal sent by external antenna 110 may simply be a power transmission signal for the purpose of locating the position of the neural stimulator 114 relative to external antenna 110 without neural stimulator 114 generating electric pulses. In other implementations, a digital signal controlled by the processor 230 may also be transmitted to the neural stimulator 114 to provide instructions (parameter-setting attributes) for the configuration of the neural stimulator 114. The digital signal may modulate, via the pulse generator circuitry 236, the carrier signal and may be incorporated into the composite signal that is transmitted to the stimulator antenna 238. In one embodiment the digital signal and powering signal are interleaved in time within the composite signal (where each signal modulates the carrier in turn, in alternating sequence). In this embodiment the data signal and powering signal may be controlled by processor 230 to have different RF power levels within the composite signal. In this embodiment, the neural stimulator 114 may process the received radiative signal 112 in a manner such that data signals are processed differently versus powering signals, and the neural stimulator 114 may be powered primarily by the powering signals. In another embodiment the digital signal and powering signal are combined into one signal, where the digital signal may be additionally used to modulate the amplitude of the RF powering signal, and thus the neural stimulator 114 is powered directly by the received radiative signal 112 without a need for separately processing data signals versus powering signals. In this embodiment, the neural stimulator 114 may extract the data content of the received signal while also harnessing the power of the received signal to power the neural stimulator 114.

The RF switch 223 may be a multipurpose device such as a dual directional coupler, which passes the RF pulses to the external antenna 110 with minimal insertion loss while simultaneously providing two outputs to the feedback subsystem 212; one output delivers a forward power signal to the feedback subsystem 212, where the forward power signal may be an attenuated version of the amplified composite signal sent to the external antenna 110, and the other output delivers a reverse power signal to a different port of the feedback subsystem 212, where reverse power may be an attenuated version of the backscatter signal 116 received by the external antenna 110. The reverse power signal, which may include backscatter signal 116 from neural stimulator 114 and/or RF signals generated by neural stimulator 114, may be processed in the feedback subsystem 212.

The feedback subsystem 212 of the transmitter 106 may include reception circuitry to receive and extract telemetry or other feedback signals from the neural stimulator 114 and/or reflected backscatter signal 116 received by external antenna 110. The feedback subsystem may include an amplifier 226, a filter 224, a demodulator 222, and an A/D converter 220.

The feedback subsystem 212 may receive the forward power signal from the RF switch 223 and may convert this AC signal to a DC level that may be sampled and sent to the controller subsystem 214. The characteristics of the forward power signal may be compared to a reference signal within the controller subsystem 214. If a disparity (error) exists in any parameter, the controller subsystem 214 may adjust the parameters affecting the amplified composite signal in the transmitter 106. The nature of the adjustment may be, for example, proportional to the computed error. The controller subsystem 214 may incorporate additional inputs and limits on its adjustment scheme such as the signal amplitude of the detected reverse power signal from the RF switch 223 and any predetermined maximum or minimum values for various operational parameters.

The reverse power signal from the RF switch 223 may, for example, be used to detect fault conditions in the RF-power transmission system of the transmitter 106. In an ideal condition, when the external antenna 110 has perfectly matched impedance to the body tissue, the radiative signal 112 generated from the transmitter 106 efficiently passes from the external antenna 110 into the body. However, in real-world applications a large degree of variability may exist in the body types of users, types of clothing worn, and positioning of the external antenna 110 relative to the body surface. Since the impedance of the external antenna 110 depends on the relative permittivity of the underlying tissue and any intervening materials, and also depends on the overall separation distance of the antenna from the skin, in any given application there may be an impedance mismatch at the interface of the external antenna 110 with the body surface. When such a mismatch occurs, the radiative signal 112 sent from the transmitter 106 is partially reflected at this interface, and this reflected energy propagates backward through the antenna feed of external antenna 110.

In one example, the RF switch 223 may be a dual directional coupler that may reduce or prevent the reflected RF signal propagating back into the amplifier 226 by attenuating the reflected RF signal while sending the attenuated signal as the reverse power signal to the feedback subsystem 212. The feedback subsystem 212 may convert this high-frequency AC signal to a DC level that may be sampled and sent to the controller subsystem 214. The controller subsystem 214 may then calculate the ratio of the amplitude of the reverse power signal to the amplitude of the forward power signal, denoted as reflected-power ratio. The ratio of the amplitude of reverse power signal to the amplitude level of forward power may indicate the severity of the impedance mismatch of external antenna 110 with the contacting body tissue.

In order to sense impedance mismatch conditions, the controller subsystem 214 may measure the reflected-power ratio in real-time. According to preset or adjustable thresholds for this measurement, the controller subsystem 214 may modify the level of the amplified composite signal generated by the transmitter 106. For example, for a moderate reflected-power ratio, the course of action may be for the controller subsystem 214 to increase the amplitude of amplified composite signal sent to the external antenna 110, as would be needed to compensate for slightly non-optimum but acceptable degree of coupling of external antenna 110 to the body tissue. For a higher reflected-power ratio, the course of action may be to prevent the transmitter 106 from generating the amplified composite signal and to set a fault code within controller subsystem 214 to indicate that the external antenna 110 has little or no coupling with the body tissue. This type of reflected-power ratio fault condition may also be generated by a poor or broken connection of the transmitter 106 to the external antenna 110. In either case, it may be desirable to stop generation of the amplified composite signal when the reflected-power ratio is above a defined threshold, because internally reflected signal power may result in unwanted heating of internal components of transmitter 106. Further, this fault condition means the external controller 101 may not be able to deliver sufficient power to the neural stimulator 114 and thus the wireless stimulation system 100 cannot deliver the intended therapy to the user.

FIG. 2C shows an example neural stimulator 114 of the wireless stimulation system of FIG. 1. FIG. 2C shows the external antenna 110 connected, via a wired or wireless connection 108, to the transmitter 106. The external antenna 110 may be fully powered by the transmitter 106. Additionally or alternatively, the external antenna 110 may include an optional battery 111 used to separately power the antenna. For example, if the external antenna 110 is wired to the transmitter 106, the signal received over connection 108 may be output as radiative signal 112 to the neural stimulator 114 without amplification. However, if the external antenna 110 receives the signal from the transmitter 106 over a wireless connection 108, then the received signal may need to be amplified before being transmitted as radiative signal 112 to the neural stimulator 114. Power for that amplification may be provided by the optional battery 111.

The radiative signal 112 may be received by a stimulator antenna 238 of the neural stimulator 114. In the example of FIG. 2C, an embodiment of the stimulator antenna 238 is shown as a dipole antenna with two poles: pole A 238A and pole B 238B. The received signal, received via stimulator antenna 238, is conveyed via respective lines to a switching circuit 256. Switching circuit 256 may be controlled by a controller 250 to permit the received signal to pass energy to the sub circuits of neural stimulator 114. The controller 250 may be comprised of discrete components and/or one or more application specific integrated circuits (ASICs). Based on a signal (or lack of a signal) from the controller 250, the switching circuit 256 may alter the connection between the stimulator antenna 238 and the stimulator sub circuits by one or more instances or permutations of shorting the connections from pole A 238A and pole B 238B to the circuit common net (or circuit “ground”) of neural stimulator 114, or alternatively to an arbitrary sub circuit of neural stimulator 114, while simultaneously creating an open circuit between the connection from pole A 238A to other stimulator sub circuits, and/or creating an open circuit between the connection from pole B 238B to other stimulator sub circuits.

The neural stimulator 114 may include one or more components that provide rectification of the AC radiative signal 112 received by the stimulator antenna 238, e.g. via a rectifier 244. In some implementations, the rectified signal may be modulated in real-time and/or conveyed directly to a charge balancer 246 that is configured to ensure that the one or more electrical pulses result in a charge balanced electrical stimulation waveform at the one or more electrodes 254. Alternatively in some implementations, the rectified signal may be conveyed directly to a controller 250, which may generate or modulate stimulus pulses (e.g., in real-time or in a programmatically delayed fashion), which are conveyed to a charge balancer 246. In some implementations, the radiative signal 112 may include encoded instructions from the transmitter 106 that control the operational parameters of the controller 250 and in such implementations the controller 250 may receive the encoded instructions via a signal tap from switching circuit 256. The pulses from controller 250, or in some implementations directly from rectifier 244, may be conveyed to a current limiter 248, whose output may be received by an electrode interface 252. The electrode interface 252 may include one or more switches or power couplings, which in some implementations, are controlled by a controller 250. In some implementations, the electrode interface 252 routes the pulses to the electrodes 254.

The current limiter 248 may be configured to limit the current level of the pulses passed to the electrodes 254 such that the current applied to the tissue does not exceed a current threshold. In some examples, the current limiter 248 may not be included, and instead the output of the charge balancer 246 may be received by the controller 250, which may use this feedback signal to control the amplitude of the current in a closed-loop fashion (including limiting the current). The independent current limiter 248 may be beneficial where, in some implementations, the amplitude of the stimulus is designed to be proportional to an amplitude (for example, current level, voltage level, or power level) of the received radiative signal 112. In these implementations, it may be beneficial to include current limiter 248 to prevent excessive current or charge being delivered through the electrodes to the tissue, although current limiter 248 may be used in other implementations. Generally, for a given electrode having several square millimeters surface area, it is the charge per phase that may be limited for safety (where the charge delivered by a stimulus phase is the integral of the current). Alternatively or additionally, in some implementations, the controller 250 may be designed to limit charge per phase. Alternatively or additionally, in some implementations the processor 230 of transmitter 106 may be configured to programmatically limit charge per phase when encoding parameter-setting attributes into the composite signal. But, in some cases, the limit may instead be placed only on the current amplitude. The current limiter 248 may automatically limit or “clip” the stimulus phase to maintain the amplitude within the safety limit.

The controller 250 of the neural stimulator 114 may control the electrode interface 252 to control various aspects of the electrode configuration pattern and pulses applied to the electrodes 254. The electrode interface 252 may act as a multiplexer and control the polarity and/or switching of each of the electrodes 254. For instance, in some implementations, the transmitter 106 may comprise multiple electrodes 254 in contact with tissue. For a given stimulus, the controller 250 may control, via electrode interface 252, one or more electrodes to 1) act as a stimulating electrode, 2) act as a return electrode, or 3) be inactive. The assignment of such an electrode pattern may be based on encoded parameter-setting attributes sent from the transmitter 106 and received and implemented by the controller 250.

In some implementations, for a given stimulus pulse, the controller 250 may control the electrode interface 252 to divide the current among the designated stimulating electrodes (e.g., one or more of electrodes 254). This control over electrode assignment and/or current control may be advantageous because in practice the electrodes 254 may be spatially distributed along various neural structures in the body. Through selection of an electrode at a given location and designation of the current amplitude for that electrode, the resulting aggregate current distribution in tissue may be shaped in order to selectively activate specific neural targets and not stimulate other neural tissues. This strategy of “current steering” may improve the therapeutic effect for the patient.

In another implementation, the shape of a stimulus waveform may be manipulated by the wireless stimulation system 100. A given stimulus waveform may be initiated and terminated at selected times, and this time course may be synchronized across all stimulating and return electrodes. Optionally, the pulse repetition rate of this stimulus cycle may be synchronous (or not synchronous) for all the electrodes. For example, controller 250, operating on its own internal algorithm or in response to encoded instructions (e.g., parameter-setting attributes) from transmitter 106, may control the electrode interface 252 to designate one or more subsets of electrodes to deliver stimulus waveforms with non-synchronous start and stop times, and the pulse repetition rate of each stimulus cycle may be arbitrarily and independently specified.

In some implementations, the controller 250 may arbitrarily shape the stimulus waveform amplitude during the course of the stimulus phase. Controller 250 may do so in response to encoded instructions (parameter-setting attributes) from transmitter 106. In some implementations, the stimulus phase may be delivered by a constant-current source. In other implementations, the stimulus phase may be delivered by a constant-voltage source. In other implementations, the stimulus phase may be delivered by a constant-power source. In general, the manner of stimulus control may generate characteristic waveform shapes that are known or static, e.g. a constant-current source generates a characteristic rectangular pulse in which the current waveform has a steep rise, then a constant amplitude for the duration of the stimulus phase, then a steep return to baseline. Alternatively or additionally, the controller 250 may increase or decrease the level of current (or voltage or power) at any time during the stimulus phase. Thus, in some implementations, the controller 250 may deliver arbitrarily shaped stimulus waveforms such as a triangular pulse, sinusoidal pulse, or Gaussian pulse for example. Similarly, the charge-balancing phase may be amplitude-shaped as desired. Similarly, in some implementations, a leading anodic pulse (prior to the stimulus phase) may also be amplitude-shaped.

As described above, the neural stimulator 114 may include a charge balancer 246. In some implementations, a controller 250, e.g., without a separate charge balancer component, may be configured to ensure the stimulus waveform has a net zero charge. In either implementation, charge-balanced stimulus waveforms are generated by design because biphasic, charge-balanced stimuli are thought to have minimal damaging effects on tissue by reducing or eliminating electrochemical reaction products that may result from driving electrical charge through the electrode-tissue interface at electrodes 254.

In some implementations, the charge balancer 246 may use one or more DC-blocking capacitors in series with the stimulating electrodes and body tissue. In a multi-electrode neural stimulator, one or more charge-balance capacitors may be used for each electrode or one or more centralized capacitors may be used within the stimulator circuitry prior to the electrode interface 252. The stimulus waveform created prior to the charge-balance capacitor (referred to as a “drive waveform”) may be controlled such that its amplitude is varied during the duration of the drive pulse. The shape of the stimulus waveform may be modified in this fashion to create a physiologically advantageous stimulus.

In some implementations, the neural stimulator 114 may create a drive-waveform envelope that follows the envelope of the radiative signal 112 received by the stimulator antenna 238. In this case, the transmitter 106 may directly control the envelope of the drive waveform within the neural stimulator 114, and thus no energy storage may be required inside the neural stimulator itself. In this implementation, the stimulator circuitry may modify the envelope of the drive waveform or may pass it directly to the charge balancer 246.

In some implementations, the neural stimulator 114 may deliver a single-phase drive waveform to the charge balancer 246 or it may deliver multiphase drive waveforms. In the case of a single-phase drive waveform, the pulse comprises the physiological stimulus phase, and the charge balancer 246 may be polarized (charged) during this phase. After the drive pulse is completed, the charge balancing function is performed by charge balancer 246, where due to the polarization resulting from the stimulus phase the accumulated charge is discharged through the tissue (driven in the opposite sense relative to the stimulus phase). In some implementations, a resistor within the neural stimulator facilitates the discharge of the charge balancer 246.

In the case of multiphase drive waveforms, the neural stimulator 114 may perform internal switching via an electrode interface 252 to pass negative-going or positive-going pulses (phases) to the charge balancer 246. These pulses may be delivered in any sequence and with varying amplitudes and waveform shapes to achieve a desired physiological effect.

In some implementations, the amplitude and timing of stimulus and charge-balancing phases is controlled by the amplitude and timing of RF pulses from the transmitter 106, and in other implementations this control may be administered internally by a controller 250. In the case of onboard control, the amplitude and timing may be specified or modified by parameter-setting attributes sent from the transmitter 106.

In some implementations a controller 250 may determine whether an input power level from the rectifier 244 is above a power threshold. Based on the determination, the controller 250 may selectively control the switching circuit 256 to reduce the power received by the neural stimulator 114 from radiative signal 112. The control of the switching circuit 256 to reduce the power may be achieved by preventing the received power from being conveyed to the rectifier 244 in accordance with a ratio of the switching circuit 256 being in one state compared to another. For example, if a power level received at the neural stimulator 114 was 100 mW and the neural stimulator only requires 90 mW, the controller 250 may cycle the switching circuit 256 with a 90% duty cycle (permitting current flow for 90% of the time and stopping current flow for 10%) of the time. The result would reduce the power received to at or below the 90 mW threshold.

This ability of the controller 250 to selectively short, open, or otherwise modify the incoming power from radiative signal 112 may be used in conjunction with other operations of the neural stimulator to provide feedback to the transmitter 106 via the backscatter signal 116. For instance, when a power level from the received radiative signal 112 is within an operating range required by neural stimulator 114, the controller 250 may control the switching circuit 256 to change the RF impedance of the stimulator antenna 238, such that the change in the RF impedance may be detected by the external controller 101 via the backscatter signal 116. When the transmitter 106 determines a cycling of the RF impedance of the neural stimulator 114, the transmitter 106 may adjust its operation accordingly. For example, in the above situation where the neural stimulator is attempting to reduce the power it receives by 10%, the transmitter 106 may in response reduce its power output until the cycling of the RF impedance of the neural stimulator 114 is no longer occurring. Alternatively or additionally, the transmitter 106 may for example determine that the duty cycle of the RF impedance variation of neural stimulator 114 is 90% and, in response, the transmitter 106 may reduce the power of radiative signal 112 by 10%.

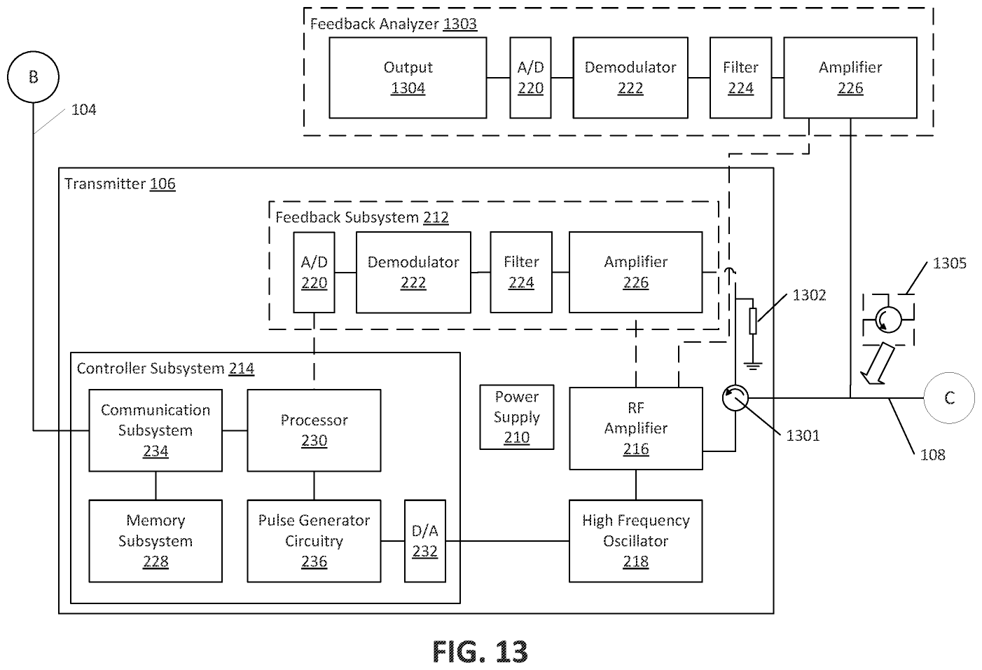

If the neural stimulator 114 provides no response to the incoming radiative signal 112 from the external controller 101, then the power provided by the transmitter 106 may be too low for the neural stimulator 114 to operate. When the power of radiative signal 112 is sufficient to operate neural stimulator 114, a positive response (e.g., that a minimum power is being received) from neural stimulator 114 may include the neural stimulator 114 alternating its impedance (where the alternating impedance may be subsequently detected as a modulated backscatter signal 116 by the transmitter 106 and/or a feedback analyzer 1303 described in relation to FIG. 13 below). The backscatter signal 116 may comprise a reflection of radiative signal 112 (e.g., due to the neural stimulator 114 varying its RF impedance at 32 ms intervals or thereabouts). Further, the backscatter signal 116 may encode a pattern generated by controller 250.

The existence of the alternating backscatter signal 116 may be determined by the external controller 101 measuring the power of the backscatter signal 116 received by the external antenna 110. The change in backscatter power is provided by the change in RF impedance of the stimulator antenna 238. In one example, the neural stimulator 114 may have two thresholds at which the controller 250 operates the switching circuit 256 to modify the RF impedance of the stimulator antenna 238: a first threshold when the neural stimulator 114 receives enough power to start operating and a second threshold when the neural stimulator 114 receives excess voltage. When the first threshold is satisfied, the controller 250 may control the switching circuit 256 to change the RF impedance of the stimulator antenna 238 (e.g., by shorting the connections from pole A 238A and from pole B 238B). When the second threshold is satisfied, the controller 250 may control the switching circuit 256 to change the radio frequency impedance of the stimulator antenna 238. How the switching circuit 256 changes the RF impedance may be the same when either threshold is satisfied. Alternatively, how the switching circuit 256 changes the RF impedance may be different when either threshold is satisfied. For example, when the first threshold is reached, the controller 250 may control the switching circuit 256 to perform one of shorting the connections between pole A 238A and pole B 238B or opening the connection to the rectifier 244 of one or more of pole A 238A or pole B 238B. When the second threshold is reached, the controller 250 may control the switching circuit 256 to perform the other of shorting or opening the connections. Additionally or alternatively, the timing associated with the controller 250 controlling the switching circuit 256 may be the same for when either of the first threshold or the second is reached or may be different for each threshold. For instance, the controller 250 may control the switching circuit 256 to modify the RF impedance of the stimulator antenna 238 at one duty cycle when the first threshold is satisfied and at another different duty cycle when the second threshold is satisfied. Alternatively or additionally, the controller 250 may control the switching circuit 256 to modify the RF impedance of the antenna 238 a first number of times per second when the first threshold is satisfied and at another number of times per second when the second threshold is satisfied.

The radiative signal 112 received by the stimulator antenna 238 may be conditioned into waveforms that are controlled within the neural stimulator 114 by a controller 250 and routed by an electrode interface 252 to electrodes 254 that are placed in proximity to the tissue to be stimulated. In some implementations, the neural stimulator 114 contains between two to sixteen electrodes 254. In yet further implementations, the number of electrodes may be over sixteen electrodes 254.

The controller 250 of the neural stimulator 114 may transmit informational signals, such as a telemetry signal, through the stimulator antenna 238 to communicate with the transmitter 106. For example, the telemetry signal from the neural stimulator 114 may be coupled to its stimulator antenna 238. The stimulator antenna 238 may be connected to electrodes 254 in contact with tissue to provide a return path for the transmitted signal. An A/D (not shown) converter may be used to transfer stored or real-time data to a serialized pattern that may be transmitted from the stimulator antenna 238 of the neural stimulator 114. The A/D converter may be incorporated into controller 250.

A telemetry or feedback signal from the neural stimulator 114 may include stimulus parameters such as the power or the amplitude of the current that is delivered to the tissue from the electrodes. The telemetry or feedback signal may be transmitted to the transmitter 106 to indicate the strength of the stimulus waveform by means of coupling the signal to the stimulator antenna 238, which radiates the telemetry signal to the external controller 101. The feedback signal may include either or both an analog and digital telemetry pulse modulated carrier signal. Data such as stimulation pulse parameters and measured characteristics of neural stimulator performance may be stored in an internal memory device within the neural stimulator 114 and may be sent via the telemetry signal. The frequency of the carrier signal may be in the range of at 300 MHz to 8 GHz (preferably between about 700 MHz and 5.8 GHz and more preferably between about 800 MHz and 1.3 GHz).

In the feedback subsystem 212, the telemetry signal may be down-modulated using demodulator 222 and digitized through an analog-to-digital (A/D) converter 220. The digital telemetry signal may then be routed to a processor 230 for interpretation. The processor 230 of the controller subsystem 214 may compare the reported stimulus parameters to those held in memory 228 to verify the neural stimulator 114 delivered the specified stimuli to tissue. For example, if the wireless stimulation device reports a lower current than was specified, the power level of radiative signal 112 from the transmitter 106 may be increased so that the neural stimulator 114 will have more available power for stimulation. The neural stimulator 114 may alternatively generate telemetry data in real-time, for example, at a rate of 8 Kbits per second. All feedback data received from the neural stimulator 114 may be logged against time and sampled to be stored for retrieval to a remote monitoring system accessible by the health care professional.

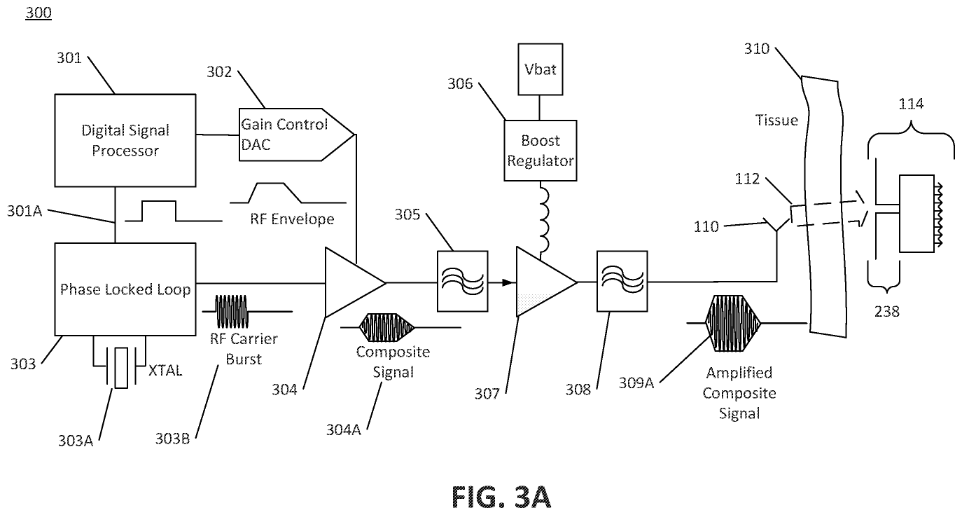

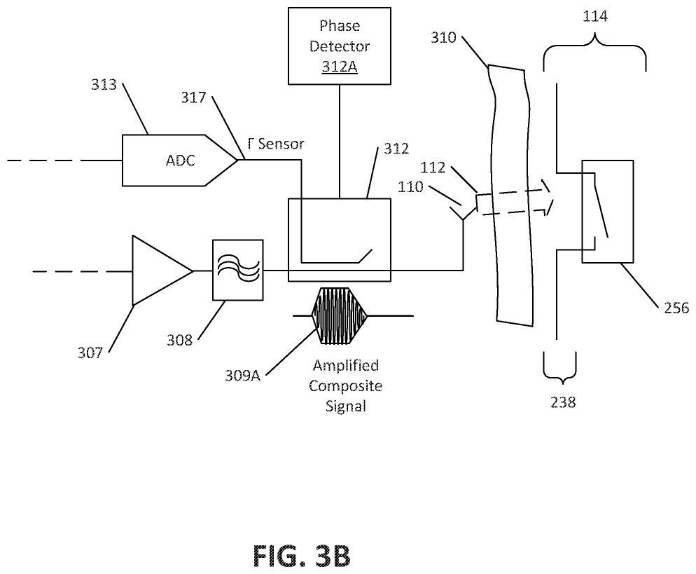

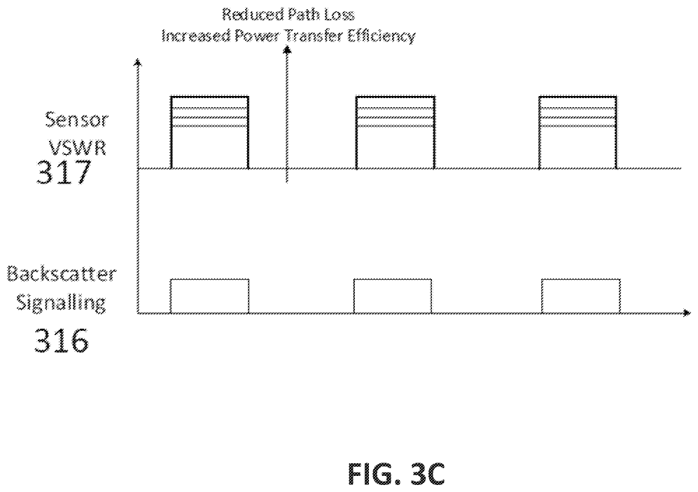

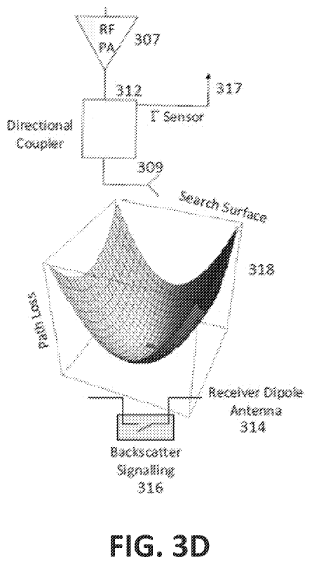

Referring to FIGS. 3A to 3D, some implementations may use the transmitter 106 for wireless power transfer, as shown in system level diagram 300. FIG. 3A is an example of one implementation of the functional blocks included in the transmitter for generating the pulsed RF signals described prior: the composite signal, amplified composite signal, and radiative signal 112 for wireless power transfer to a neural stimulator 114. The transmitter may include a digital signal processor 301, a gain control 302, a phase-locked loop 303, a gating amplifier 304, a pulse-amplitude input matching network 305, a boost regulator 306, a radio-frequency (RF) amplifier 307, a pulse-amplitude harmonic filter 308. A connected external antenna 110 may be placed near or in contact with a tissue boundary 310. The transmitter generates the radiative signal 112 in the body tissue to energize the neural stimulator 114.

Digital signal processor 301 may generate pulse parameters such as pulse width, amplitude, and pulse repetition rate. Digital signal processor 301 may feed pulse parameters to a gain control 302, which can include a digital-to-analog converter (DAC). Gain control 302 may generate RF envelope 302A to gating amplifier 304. Digital signal processor 301 may feed the phase-locked loop 303 with stimulus timing control 301A, which is a voltage signal that drives crystal XTAL 303A to generate RF carrier burst 303B. RF carrier burst 303B arrives at gating amplifier to modulate RF envelope 302A such that composite signal 304A is generated to feed the pulse-amplitude input matching network 305.

Output from pulse-amplitude input matching network 305 is provided to RF amplifier 307 under a bias voltage from boost regulator 306. Subsequently, a harmonic filter 308 mitigates harmonic distortions and feeds the filtered output as amplified composite signal 309A to the transmitter antenna 1109. The radiative signal 112 is transmitted from the external antenna 110 through the tissue boundary 310 to reach neural stimulator 114.

FIG. 3B is an example of one implementation of the functional blocks included in a transmitter for monitoring the amplified composite signal 309A. A directional coupler 312, and an analog-digital converter (ADC) 313. Monitoring the amplified composite signal may be used for closed-loop control of the signal by the transmitter for example.

In some implementations, the stimulator antenna 238 and external antenna 110 may exhibit mutual coupling. In some implementations, the mutual coupling of the stimulator antenna 238 and external antenna 110 may be monitored by external controller 101 for the purpose of assessing the state of the neural stimulator 114.

In some implementations an estimated geometric factor may be included in the measurement normalization that may account for the change in mutual coupling for various thicknesses of tissue that separates the stimulator antenna 238 from the external antenna 110.

Some implementations incorporate RF complex impedance measurement via F Sensor subsystem, which may include an RF phase detector 312A, shown in FIG. 3B. The backscatter signal 116 is received at external antenna 110 and routed via directional coupler 312 to analog-digital converter (ADC) 313, and from this signal the RF impedance, or reflection coefficient, may be calculated.

In some implementations, the wireless stimulation system 100 may use the RF reflection measurements to obtain the electrode-tissue impedance at the interface of one or more electrodes 254 with the body tissue. At the location in the patient where the neural stimulator is placed, the extracellular environment around the electrodes may change due to insertion-related damage and the presence of the electrodes (foreign material) in the tissue, both of which may instigate formation of scar tissue, a compact sheath of cells and extracellular matrix surrounding the neural stimulator. Some studies have found that this encapsulating tissue may alter electrical impedance relative to normal (or unscarred) tissue. Since a change of the electrode-tissue impedance may alter the effectiveness of the neural stimulator 114, it may be advantageous for the wireless stimulation system 100 to have the capability of assessing the impedance of the electrode-tissue interface.

In some implementations, based on the obtained electrode-tissue impedance of one or more electrodes 254 at the electrode-tissue interface, the strategy for stimulation by the wireless stimulation system 100 may be modified to compensate for the electrode-tissue impedance. For example, if the electrode-tissue impedance is found to be higher than a threshold, the wireless stimulation system 100 may compensate by affecting higher voltage output or current output for the neural stimulator 114.

As discussed in detail through FIGS. 1 and 2A-2C, the radiative signal 112 is received by the stimulator antenna 238 and subsequently rectified via a rectifier 244. In some implementations, the received energy is stored in a capacitor in the neural stimulator 114. In general the energy stored in the capacitor is a function of the charge held by the capacitor and the voltage across the capacitor. In some implementations, the stored charge is used for smoothing the rectified signal from rectifier 244 and/or short-term supply of energy to circuits within neural stimulator 114. In some implementations, the stored charge can be used for transient energy needs of controller 250, for example to generate a stimulus waveform. Thus, it may be useful for the external controller 101 to determine the level to which the storage capacitor is charged.

In some implementations, the signal received at the F Sensor of FIG. 3B is processed to deduce the state of charge of the storage capacitor in the neural stimulator 114. In more detail, the time-varying currents and voltages at the rectifier and capacitor act to create a variable RF impedance at the feed point of stimulator antenna 238. When the capacitor has low charge, the RF current flows freely to rectifier 244, which is a near RF short circuit at the antenna's feed point. When the capacitor approaches full charge, the RF current from the antenna's feed point is impeded, such that there is a near RF open circuit at the feed point. It follows that the complex impedance at the feed point of stimulator antenna 238 is an indicator of the state of charge of the capacitor in the neural stimulator 114. Because the dynamic impedance at the stimulator antenna is coupled to the external antenna 110, the impedance at the stimulator antenna may be observed by the F Sensor of FIG. 3B, and from this measurement, the external controller 101 may deduce the state of charge of the storage capacitor in the neural stimulator 114.

In some implementations, the radiative signal 112 may be judiciously selected to maintain the state of charge of the storage capacitor in the neural stimulator 114 at a desired, constant level. For example, the radiative signal 112 pulse rate and width may be strategically selected to maintain a steady-state delivery of power to the neural stimulator 114 such that energy is delivered at the same rate that it is consumed by the stimulator circuitry.

In some implementations, the state of charge of the capacitor in the neural stimulator 114 is an indicator of the neural stimulator present operational state and environment. The voltage at the capacitor will decay proportionally to the rate at which energy is depleted by the load connected to the capacitor. The load may encompass the load at the stimulator's electrodes (tissue) and the load of the circuitry associated with transferring charge from the capacitor to the electrodes 254.

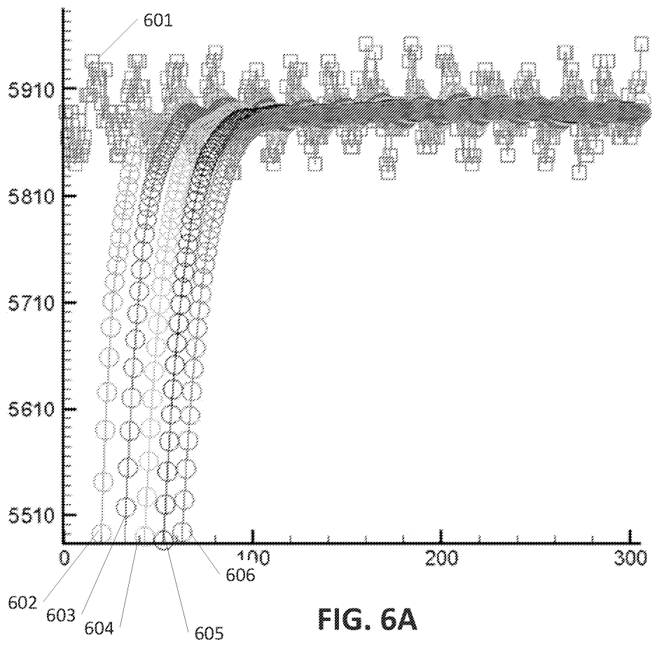

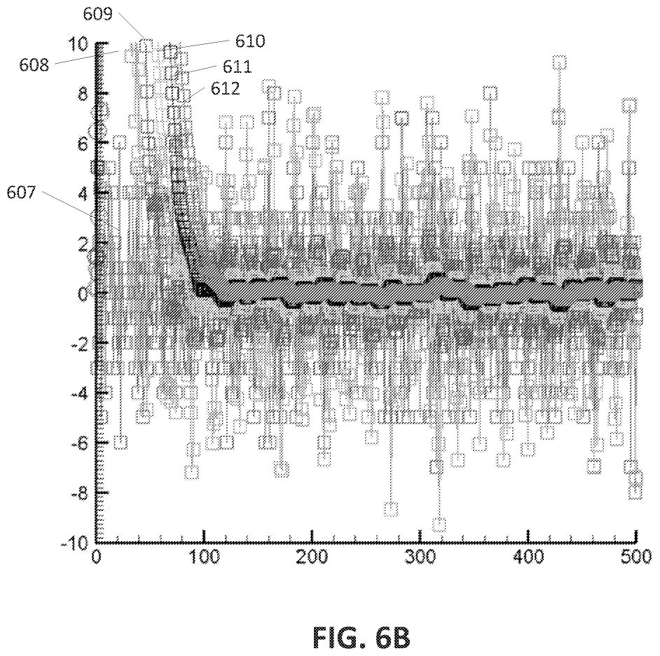

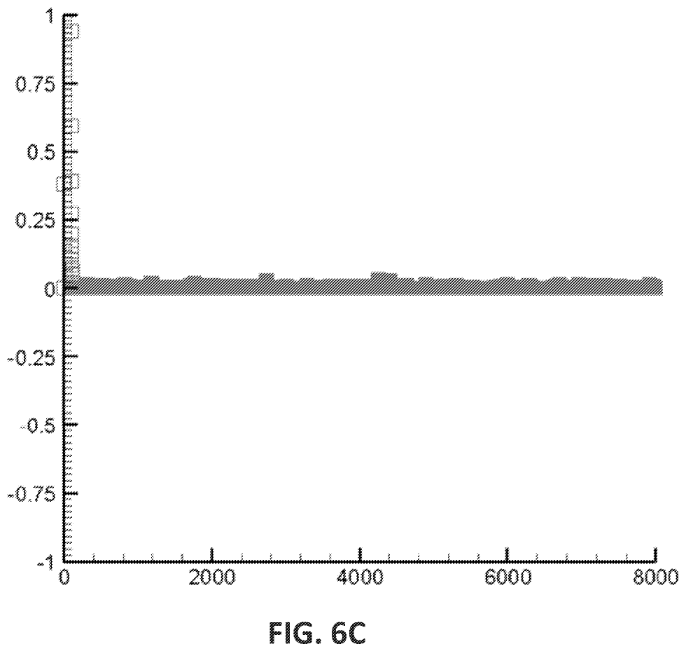

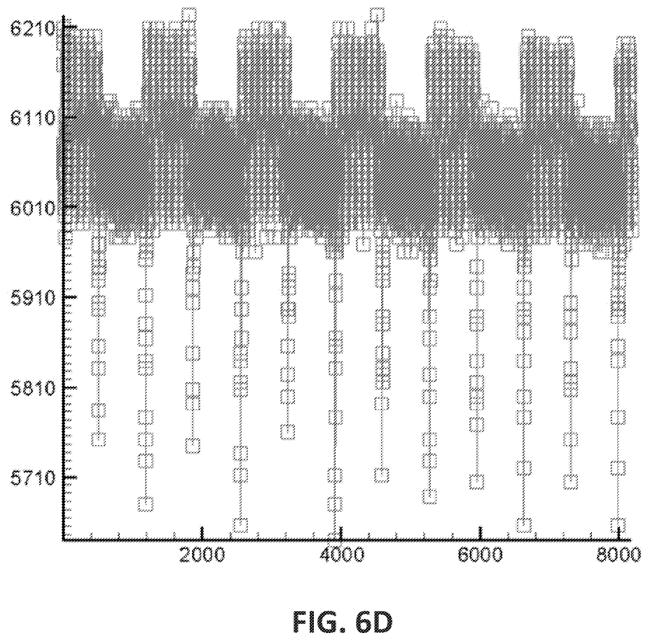

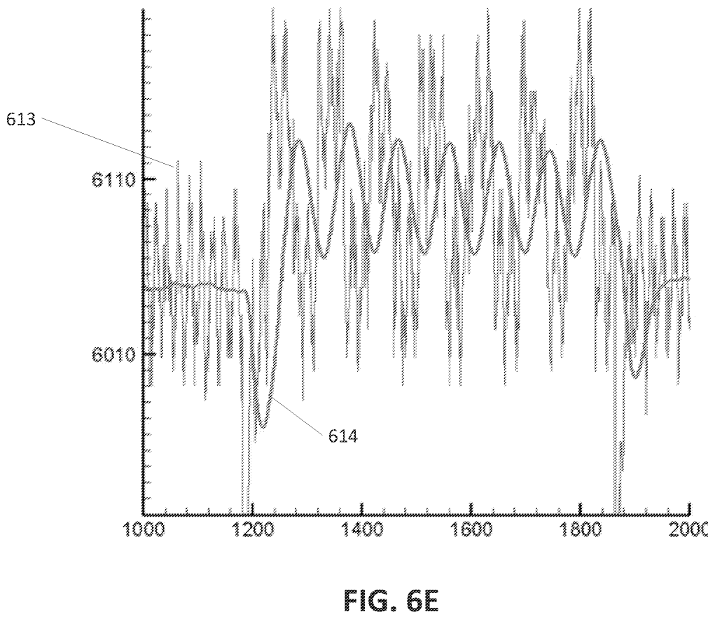

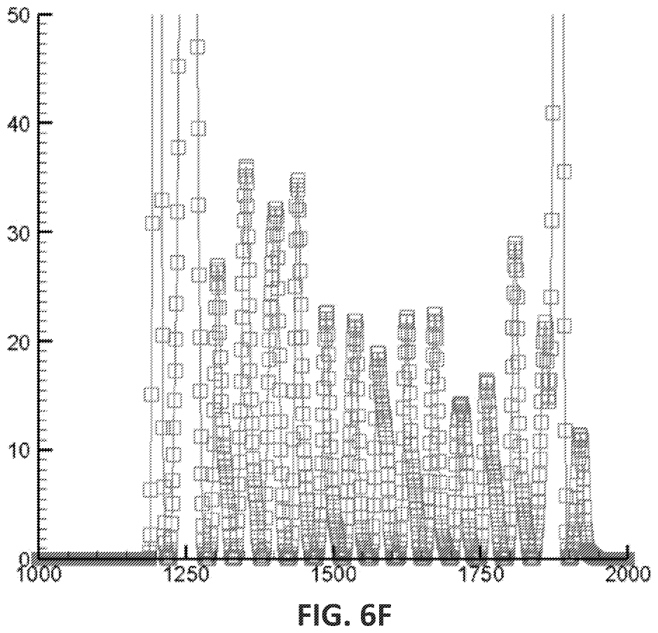

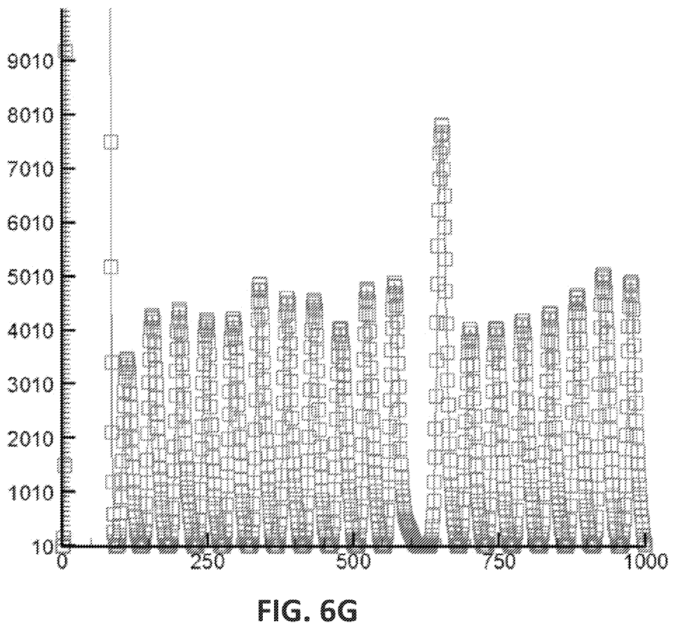

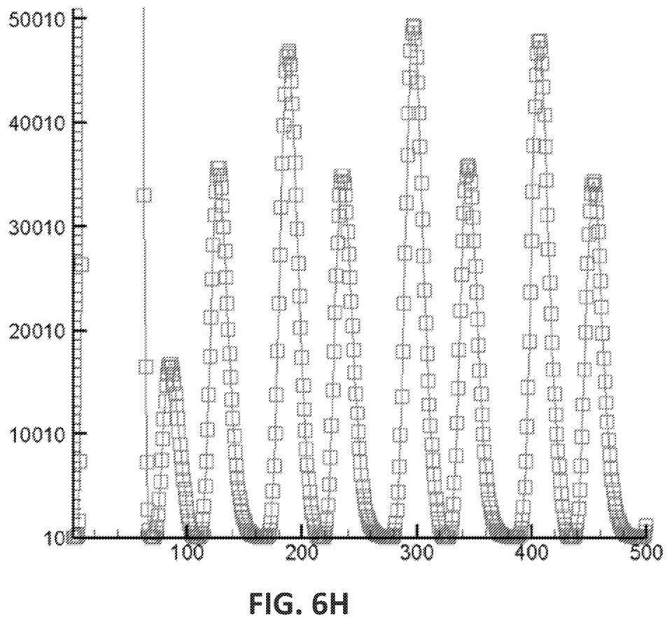

In some implementations, the rate at which charge is depleted from the capacitor in the neural stimulator 114 depends on the stimulus parameters, the electrode-tissue impedance, and the internal circuitry of the neural stimulator. By virtue of such dependence, the rate of charge depletion from the capacitor may be used to determine the electrode-tissue impedance of the electrode-tissue interface. The rate of charge depletion may reveal an RF impedance characteristic of the stimulator antenna 238 from which the electrode-tissue impedance may be extracted. For example, if the electrode-tissue impedance is mostly resistive and is sufficiently low (for example, z may range between 300 and 500Ω), the intended stimulus current will be driven to the targeted tissue, and the charge on the capacitor will deplete at an expected rate. In contrast, if the electrode-tissue impedance has a high-value resistance or is dominated by series-capacitance, the intended stimulus current may not be delivered. An example of high-value resistance is demonstrated in FIG. 6D. If the programmed stimulus current is not delivered to the tissue, the charge on the capacitor will deplete at a lower rate than expected. In both cases, the rate of charge depletion may reveal the RF impedance characteristic of the stimulator antenna 238, and from this rate a deduction about the electrode-tissue impedance of the electrode-tissue interface may be made.

In some implementations, a circuit internal to the neural stimulator 114 may allow connection of a voltage-driver or current-driver circuit to a calibrated internal load. In some implementations, a calibrated internal load in the neural stimulator 114 may be programmed to specific impedance values.

In some implementations, the neural stimulator 114 may drive voltage or current into the calibrated internal load while either the drive or the load is swept through a range of values, and the corresponding family of unique complex RF reflection coefficients may be captured for reference. Subsequently, when the neural stimulator is configured to drive voltage or current through the electrode-tissue impedance, which is unknown, the RF reflection coefficient curve may be captured and compared to the family of reference curves. By matching the curve of the unknown electrode-tissue impedance to the curve of a known load, the electrode-tissue impedance may be deduced.

In some implementations, a circuit internal to the neural stimulator 114 may facilitate a system self-check to ascertain the suitability of the wireless stimulation system 100 to provide stimulation therapy. For example, for a system self-check, the neural stimulator may drive various currents into an internal load, and for each current level the average RF power is swept while the RF reflection is observed. These measurements may be used for a reference to compare to electrode-tissue impedance measurements during the self-check.

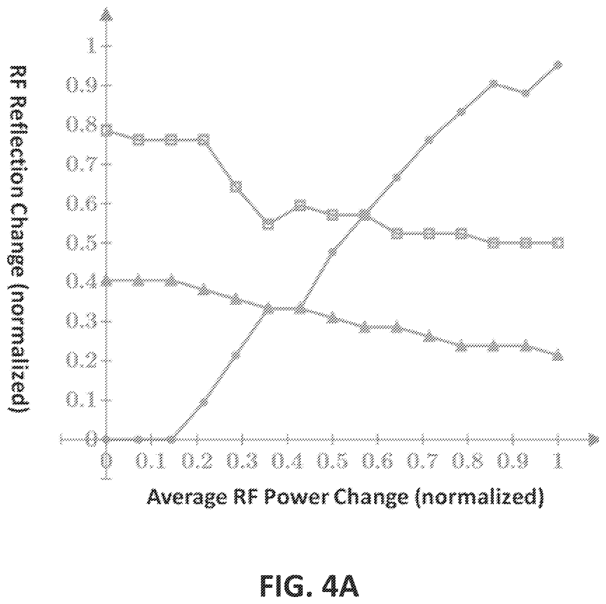

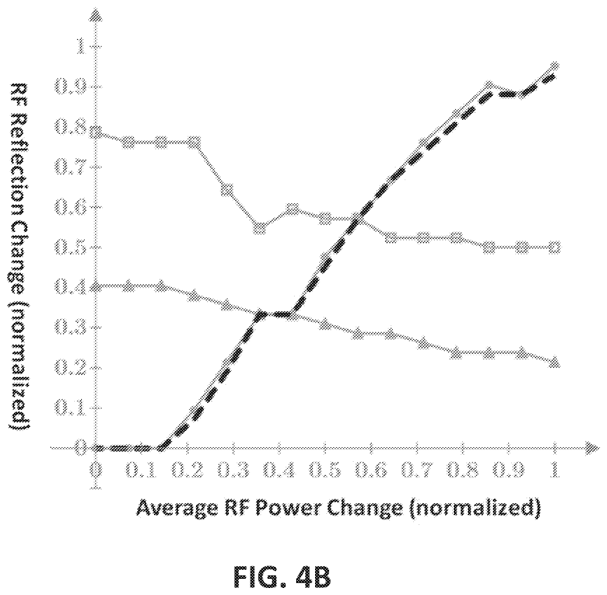

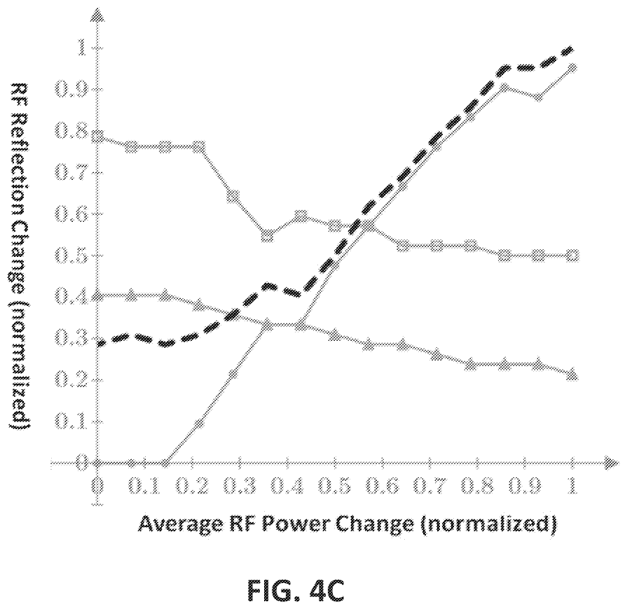

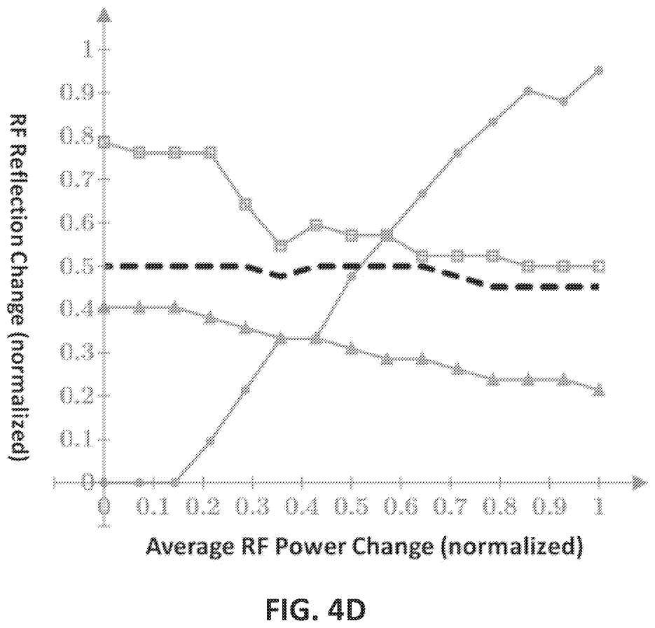

For purpose of the analysis it may be advantageous to view the change of the measurand (RF reflection, or reverse RF voltage) relative to its initial value, and the change in the independent variable (average RF power) relative to its initial value. For example, for measurand V, the change would be (V−Vmin). Further, it may be advantageous to normalize the data. The data shown in FIG. 6A through 4B was normalized to the reference measurements as follows: The maximum and minimum RF reflections (reverse RF voltages, Vmax and Vmin) of these reference measurements were extracted, and the difference was used as the normalization factor. For example, the subsequent measurand changes were normalized as follows:

v=(V−V min)/(V max−V min), (1)

where V is the measured reverse RF voltage, and v (lower case) implies normalized.

Similarly the independent variable changes were normalized as follows:

p=(P−P min)/(P max−P min), (2)

where P is the average transmitted RF power and p (lower case) implies normalized.

Some implementations incorporate location detection of the neural stimulator 114 via F Sensor subsystem of FIG. 3B. The F Sensor (“gamma” sensor or “reflection” sensor) is used to measure the voltage standing wave ratio (VSWR), from which the return loss (RL) is computed. The return loss of the RF signal can be exploited to detect a backscatter signal 116 that is modulated by the neural stimulator 114. The backscatter signal 116 is received at the external antenna 110 and routed via directional coupler 312 to analog-digital converter (ADC) 313 so that an estimate of VSWR 317 may be obtained. The graph of FIG. 3C shows how the estimate of VSWR 317 may lead to a reduced path loss and an increase in efficiency.

The location detection method can be used to determine the most advantageous position for the transmitter antenna 110, thereby minimizing the path loss from the external antenna 110 to the receiver antenna 238. The operation of searching for the neural stimulator 114 is premised on the neural stimulator 114 modulating the radio frequency impedance of its internal antenna 238, thereby modulating the backscatter signal 116. This modulation of backscatter signal 116 is detectable by the F Sensor of FIG. 3B, where F is the reflection coefficient (or return loss) of the external antenna 110. When the neural stimulator device 114 is configured to operate in location mode, the RF impedance of internal antenna 238 is periodically modified by a switched load. When the backscatter signal 116 changes, the RF impedance of the internal antenna 238 is altered such that the internal antenna 238 reflects RF energy. Subsequently, the external antenna 110 also experiences an impedance change, which is detected by the F Sensor of FIG. 3B. The measurements at the F Sensor represent the forward and reverse RF power levels, from which F is computed. As the backscatter signal 116 at the internal antenna 238 is actively modulated by the neural stimulator 114 by modulating the radio frequency impedance of the neural stimulator 114, the shift of the signal seen by the F Sensor has an observed magnitude. The magnitude of the shift also depends on the coupling of the two antennas. As the external antenna 110 is brought closer to the internal antenna 238, the RF coupling improves, and the magnitude of signal from the F Sensor increases. For example, the neural stimulator device 114 may be instructed to enable the location-setting mode based on, for instance, parameter-setting attributes contained in the composite signal from the transmitter 106. Additionally or alternatively, the location-setting mode may be enabled by the neural stimulator 114 itself based on, for example, when initially receiving enough power to enable operations of the controller 250.

When the system is engaged in location mode, the feedback subsystem 212 monitors the reflection coefficient Γ and computes the associated Voltage Standing Wave Ratio (VSWR) according to the following equation:

$$V S W R = \frac{1 + \vert Γ \vert}{1 - \vert Γ \vert} \qquad ( 3 )$$

The path loss decreases (the power transmission improves) as the external antenna 110 is moved into better alignment with the internal antenna 238. As represented by the concave 3-D surface showing the path loss versus antenna alignment in FIG. 3D, the optimal location of the external antenna 110 corresponds to the minimum value of the path-loss surface. Finding the low point on the path-loss surface is the goal of the user while moving the external antenna 110 across the surface of subject's body. While operating in this mode, the transmitter 106 may provide audio and/or visual and/or haptic feedback to the user indicating when the external antenna 110 is approaching the optimal alignment. By the use of this location determination method, the path loss for the RF power can be substantially minimized, meaning the transmitter 106 provide the most efficient power delivery to the neural stimulator 114.

In some implementations, the location determination algorithm employs a finite impulse response (FIR) filter for reducing noise from the F Sensor. By computing the summation (SUM) of F values from the most recent N pulses, then removing the baseline offset by taking the time derivative of the smoothed data, the backscatter transitions or “steps” of F can be extracted from a noisy signal. In this application, it may be advantageous to resolve small steps of F because the influence of the internal antenna 238 upon the value of F (measured at the external antenna 110) can be very small relative to the noise.

The backscatter transitions in the time derivative of F can be enhanced by raising the result to an M-th power, where M is positive and even, such that any derivative value less than 1.0 can be reduced to approximate zero, while any value above 1.0 can be enhanced. An example of a computationally efficient algorithm to perform the described signal conditioning is as follows:
Let N be an integer greater than 1, where N is the number of points or “taps” of the FIR filter.
Calculate the sums: Sum0, Sum1, Sum2, . . . , Sum5 and corresponding time derivatives, Delta0, Delta1, Delta2, . . . , Delta5 of the received signal which, in this example, is the unprocessed reverse voltage values (REV(n)) as sampled at the transmitter reverse voltage detector.
u1=Sum0
Sum0=REV(n)
u2=Sum0
Delta0=u2-u1
u1=Sum1
Sum1=(Sum1+Sum0−Sum1/N)
u2=Sum1
Delta1=u2−u1
u1=Sum2
Sum2=(Sum2+Sum1/N−Sum2/N)
u2=Sum2
Delta2=u2−u1
u1=Sum3
Sum3=(Sum3+Sum2/N−Sum3/N)
u2=Sum3
Delta3=u2−u1
u1=Sum4
Sum4=(Sum4+Sum3/N−Sum4/N)
u2=Sum4
Delta4=u2−u1
u1=Sum5
Sum5=(Sum5+Sum4/N−Sum5/N)
u2=Sum5
Delta5=u2-u1

When calculating successive sums, a divide-by-N operation may be added in order to avoid generation of very large numbers in the computations. The number of data sample points averaged, N, can be chosen strategically to remove random noise and/or known periodic noise signals. However, N may be chosen such that the algorithm has suitable settling time for the given application, and the filtering does not obscure the desired signal. For example, when looking for backscatter signals 116, N should be less than or equal to the number of samples per backscatter period. Otherwise the backscatter signal 116 itself can be filtered and lost. In the following examples, N=8, and RF pulse rate=3 kHz.

FIG. 4A shows the reference measurements including normalized change in RF reflection (based on a measurement of the backscatter signal 116) versus normalized change in RF average power for three different currents driven into the internal load. The curves show: 1) minimum current through the internal load (circles), 2) medium current through the internal load (triangles), 3) maximum current through the internal load (squares). In some cases, minimum may be around 0.1 mA; medium may be around 6 mA; and maximum may be around 12 mA. In the case of low current, the charge on the capacitor builds steadily higher as the average RF power is increased. This is indicated on the plot (circles) by the rising trajectory of the RF reflection. In the case of medium current, the charge on the capacitor remains relatively flat as the average RF power is increased. This is indicated on the plot (triangles) by the relatively flat trajectory of the RF reflection. For the case of high current, the charge on the capacitor remains relatively flat (but at different level) as the average RF power is increased. This is indicated on the plot (squares) by the relatively flat trajectory (at a higher level) of the RF reflection.

In some implementations, the wireless stimulation system 100 self-check may include various fault checks. For example, when the neural stimulator 114 is energized but not programmed to drive stimulus current, the RF reflection may be similar to that shown for the minimum-current case of FIG. 4A. This is shown in FIG. 4B, where the RF reflection for the un-configured neural stimulator (dashed line) is overlaid onto the plots of FIG. 4A.

Further, based on the reference measurements shown in FIG. 4A, the system may test for a fault in the stimulus-current driver (such as a broken electrode). For example, the neural stimulator 114 may be programmed to drive stimulus current through selected pairs of electrodes, and the RF reflection for each pair is compared against the reference measurements in FIG. 4A. When the neural stimulator is programmed to drive a given stimulus current through tissue, the RF reflection may be similar to that of when the same current is driven through the internal load. However, in the even that a wire has been damaged at the electrode, for example, the current through the circuit would be blocked, meaning the RF reflection would resemble the minimum-current curve of FIG. 4A. This case is shown in FIG. 4C, plotted with the reference measurements of FIG. 4A.

Further, during normal operation, the electrode-tissue impedance may be unknown, however, it will likely be within an expected range, and a fault-check may verify this condition. FIG. 4D shows the RF reflection (dashed line) when a 500Ω resistor is connected in series with the electrodes, and a medium current is delivered. The result shows the RF reflection falls within the expected range. However, for example, if the result showed the RF reflection overlaid the low-current curve, a fault would be evident.

By capturing the reflected RF signal and applying the analysis methods described herein, it is possible to measure the electrode-tissue impedance at the electrode-tissue interface. Furthermore, by measuring the electrode-tissue impedance, the system may adjust stimulus parameters to compensate, thereby maintaining the efficacy of stimulation.

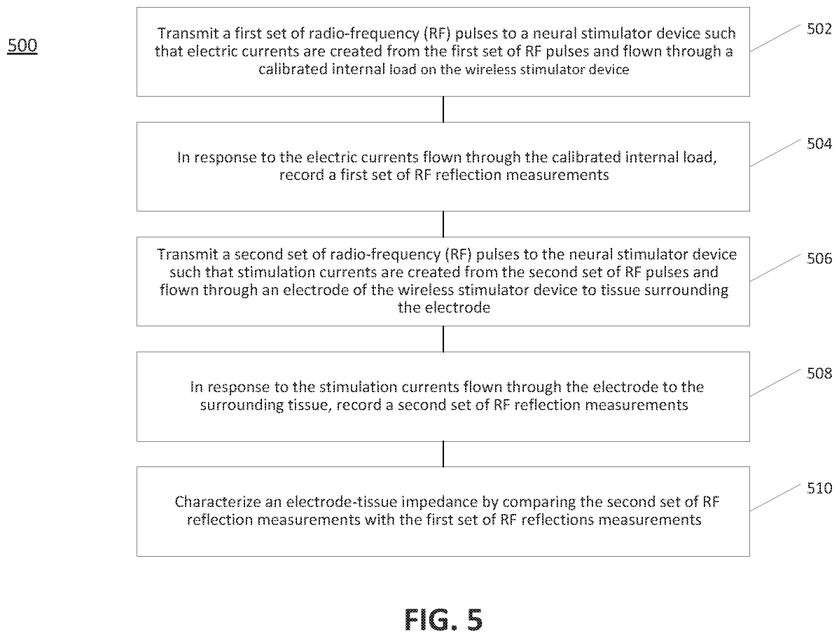

FIG. 5 shows an example of a flow chart 500 for implementing stimulation adjustment on a neural stimulator based on sensing of tissue-electrode impedance. As represented by step 502, a first set of RF pulses are transmitted from a transmitter 106 to a neural stimulator (such as 114) via non-inductive electric radiative coupling. Consistent with discussions from FIGS. 1-3D, electric currents are created from the first set of RF pulses and conveyed through a calibrated internal load on the neural stimulator. The calibrated internal load represents a load condition that is pre-determined and imposed on, for example, an electrode on the neural stimulator 114.

In response to the electric currents conveyed through the calibrated internal load, flow chart 500 proceeds to recording, on the transmitter, a first set of RF reflection measurements (504). This recording measures, for example, RF signals reflected from the neural stimulator 114 and received by transmitter 106.

Next, a second set of RF pulses are transmitted, from the transmitter and via electric radiative coupling, to the neural stimulator such that stimulation currents are created from the second set of RF pulses and provided through an electrode of the neural stimulator to tissue surrounding the electrode (506). Here, the stimulation currents flow through the stimulator circuitry, the electrode, and the electrode-tissue interface.

In response to the stimulation currents conveyed through the electrode to the surrounding tissue, a second set of RF reflection measurements is recorded on the transmitter (508). This second set of reflection measurements are based on RF signals reflected from the neural stimulator 114 and received by transmitter 106.

By comparing the second set of RF reflection measurements with the first set of RF reflections measurements, an electrode-tissue impedance is characterized (510). When the electrode-tissue impedance is characterized as resistive, one or more input pulses to be transmitted by the transmitter to the neural stimulator may be adjusted such that stimulus currents created from these input pulses on the neural stimulator are likewise adjusted to compensate for a resistive electrode-tissue impedance. When the electrode-tissue impedance is characterized as capacitive, one or more input pulses to be transmitted by the transmitter to the neural stimulator may be adjusted such that stimulus currents created from these input pulses on the neural stimulator are likewise adjusted to compensate for a capacitive electrode-tissue impedance. Here, the adjustment of input pulses involves maintaining a steady-state delivery of electrical power to the neural stimulator such that electrical energy is extracted from the input pulses as fast as electrical energy is consumed to generate the stimulus currents with one or more pulse parameters that have been varied to accommodate the resistive electrode-tissue impedance. Such stimulus currents are delivered from the electrode to the surrounding tissue. Examples of pulse parameters include: a pulse width, a pulse amplitude, and a pulse frequency.

Based on results of characterizing the electrode-tissue impedance, a stimulation session may be automatically chosen. The selection process may include: determining input pulses to be transmitted by the transmitter to the neural stimulator such that stimulus currents are created on the neural stimulator and delivered by the electrode on the neural stimulator to the surrounding tissue in a manner that, for example, maintains therapy consistency despite variations in electrode-tissue impedance. In one instance, the second set of RF pulses may be updated to obtain updated second set of RF reflection measurements; and then the updated second set of RF reflection measurements may be compared with the first set of RF reflection measurements. In this instance, the updating and comparing steps may be performed iteratively until desired RF reflection measurements are obtained.

The characterizing step may also lead to automatic fault checking according to results from such characterization. In one instance, automatic fault checking includes automatic detecting a damaged wire in a circuit leading to the electrode on the neural stimulator, as shown in, for example, FIG. 4C.

Referring to the structure of FIGS. 3A-3D, FIG. 6A is an example plot of the signal conditioning algorithm of the sample count (horizontal axis) with the values calculated as Sum0 through Sum5. In this display, Sum0 results are represented by red squares 601, Sum1 results are represented by green circles 602, Sum2 results are represented by dark blue circles 603, Sum3 results are represented by light blue circles 604, Sum4 results are represented by black circles 605, and Sum5 results are represented by magenta circles 606. FIG. 6B shows the respective time derivatives versus sample count (horizontal axis), for Delta0 through Delta5. In this view, Delta0 results are represented by red squares 607, Delta1 results are represented by green squares 608, Delta2 results are represented by dark blue squares 609, Delta3 results are represented by light blue squares 610, Delta4 results are represented by black squares 611, and Delta5 results are represented by magenta squares 612. The results show an increase in the settling time as the number of sums is increased. Sum0 results (red squares 601 of FIG. 6A), for example, has noise with an undesired periodic beat. In one instance, letting N=8 (eight points averaged) may filter out the beat sufficiently while minimizing settling lag. By the 5^{th} sum (magenta circles 606), the noise in the results is substantially smoothed out. If derivatives are subsequently taken and raised to an even power, the result will be a positive value. The derivatives smaller than 1.0 can be reduced, while any derivatives larger than 1.0 can be enhanced. The derivative of the 5^{th} pass, raised to the 4^{th} power, (Delta5)⁴, versus sample count (horizontal axis) is shown in FIG. 6C. In this case the backscatter signal 116 was turned off and only environmental noise was present. With this algorithm, the unwanted noise was substantially filtered out, as shown in FIG. 6C.

FIG. 6D shows a backscatter signal 116 for a neural stimulator 114 that is far from the external antenna 110, and the change in F is near the limit of detection. In FIG. 6E the same result is shown on a smaller time scale to show the signal in detail. From sample count 1,000 to 1,200, the backscatter signal 116 was turned off, then it was enabled from 1,200 to about 1,800, then it was off again to sample count 2,000. The red trace 613 is results from Sum0 which is the backscatter signal 116 superimposed on the noise. The magenta trace 614 shows results from Sum5, which is flat when the backscatter signal 116 is off and is sinusoidal when the backscatter signal 116 is on. The derivative of results from Sum5, raised to the 4th power, (Delta5)⁴, enhances the signal as shown in FIG. 6F, which demonstrates a weak backscatter signal 116 can be detected in a noisy environment. In contrast, a strong backscatter signal 116 with the same filtering algorithm is shown FIG. 6G. A stronger signal such as that of FIG. 6G may occur when the path loss from the external antenna 110 to the internal antenna 238 is minimized. Two additional examples of detection of backscatter signal 116 are shown in FIGS. 6H and 6I. These signals are considered to be difficult to resolve because the scattering intervals may be randomly timed. FIG. 6H shows the case when two neural stimulators 114 are backscattering simultaneously, and FIG. 6H shows the case when a neural stimulator 114 generates chaotic backscattering.

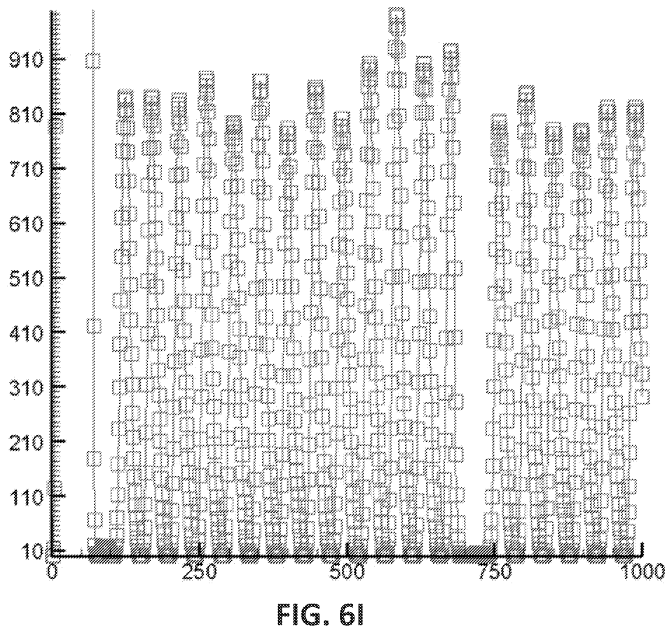

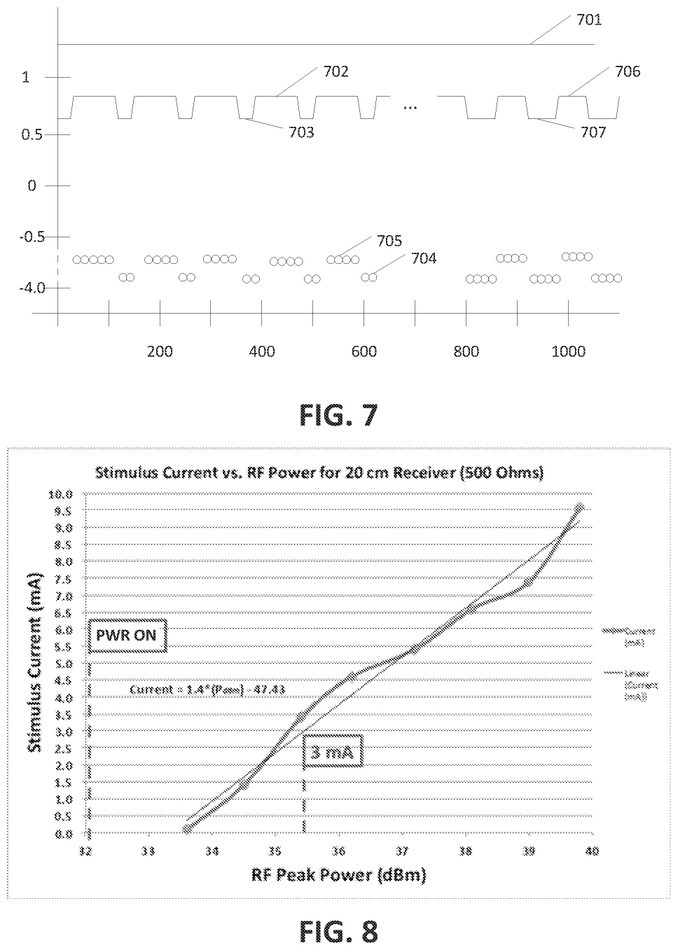

FIG. 7 is an example of power modulation of the backscatter signal 116. As the shorting or the opening of the connections from the stimulator antenna 238 may be detected by the external controller 101 as a changing radio frequency impedance as shown in FIG. 7, this may be represented with a forward voltage 701 transmitted by the external antenna 110 and a reverse voltage 702 received at the external antenna 110 where, for every 100 ms or so, the shorting/opening of the connections to the stimulator antenna 238 changes the radio frequency impedance may be sensed by the external controller 101 as lower reverse voltages 703. Dividing the lower reverse voltages 703 into the forward voltage 701 results in a reflection coefficient (represented by circles 704) (here, about 0.5 dB in signal strength). Dividing the higher reverse voltages 702 into the forward voltage results in reflection coefficient (represented by circles 705). The duty ratio of the combination of reverse voltages 702 and 703 is not 1:1. The duty ratio of a later combination of reverse voltages 706 and 707 is equal (e.g., 1:1). During the time of the reverse voltages 702 and 703, the neural stimulator may be attempting to reduce received power. During the time of reverse voltages 706 and 707, the neural stimulator may be indicating that, for instance, that a received power intensity is adequate for the operation of the neural stimulator. Further, through varying the duty cycle of the reverse voltages, different information may be communicated from the neural stimulator to the transmitter.

FIG. 8 is an example of stimulation current compared to RF power of the radiative signal 112. In an example where the stimulus current is proportional to the received RF power, the stimulus current may be increased by increasing the RF power. FIG. 8 shows an output stimulus current varying based on a supplied RF power (e.g., RF peak power in dBm).

In this example, a method for estimating power needed to produce stimulus currents for a given stimulation scenario is described. The Principal of Linearity is invoked: Specifically, with the assumption the transmitter and the neural stimulator are both operating in their respective linear regions (no FET or capacitor saturation), then any RF voltage/current level may be scaled (for increase or decrease) with the square root of the corresponding power scale. That is, to increase/decrease an RF voltage/current level with a scaling factor α, the RF power may be increased/decreased by a scaling factor of α2. In this example, measurements were taken while sweeping the RF power amplitude while allowing the stimulus current amplitude to increase freely until reaching 10 mA. As a reference, the initial power level for the neural stimulator to turn on was approximately where the RF Peak Power was around 32 dBm.

These results show an average slope in the RF power of 1.0 dB per increase of 1.4 mA of stimulus current amplitude. The oscillation about the straight line may be attributed to non-linearity of the transmitter-stimulator system. The results are generally linear. Accordingly, an initial value and slope of the curve may be used to predict a power level needed for a stimulus current value, assuming all other variables remain relatively constant.

For any given system, this curve may be repeated. There are several variables to consider, however. For example, at a fixed RF pulse rate, the slope of this curve may be dependent on the ratio of the stimulus current's total width and the RF pulse width. Also, the x-intercept of its curve may be dependent on the stimulation scenario, including separation and alignment, etc. of the external antenna and the stimulator antenna relative to each other.

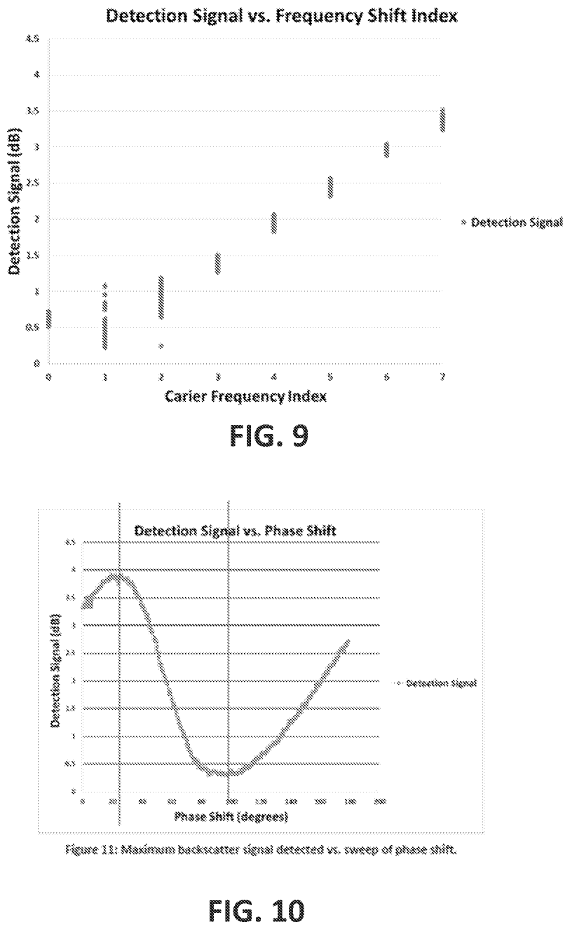

There may be conditions such that the signal from the neural stimulator is lost due to constructive interference of the carrier RF field that may be superimposed on the backscatter signal 116. For such a case, a change in the RF carrier frequency (or carrier frequency perturbation) may help with the reduction of signal interference. The carrier frequency may be automatically adjusted in, for example, the high frequency oscillator 218. The frequency may be adjusted continuously or in steps. An example of a detection signal over a stepped frequency is shown in FIG. 9 about a center frequency. For instance, an eight step frequency index may be centered on carrier frequency. As shown in FIG. 9, the steps are at 3.5 MHz around a nominal carrier center frequency. A maximum backscatter signal 116 may be detected in sweeps of approximately 320 ms vs the carrier frequency index. In response to detecting the maximum backscatter signal 116, the high frequency oscillator 218 may be adjusted to change the carrier frequency to be closer to or match the carrier frequency that produced a higher (or highest) detected backscatter signal 116. Using a 915 MHz initial carrier frequency, the steps may be increased by 3.5 MHz over a range. At 3.5/915, the resulting percent difference per step may be 0.38%. The above method of sweeping a carrier frequency through a range of frequencies (continuously or discretely) may be performed by a user. Alternatively or additionally, the method may be performed by the system (e.g., controlled by control subsystem 214) upon first finding the neural stimulator and/or periodically and/or when communication with the neural stimulator is reduced or lost.

In the event that the carrier frequency perturbation does not produce sufficient signal to noise ratio for sensing some signals, it may be beneficial to also include a phase change. A programmable phase shifter may provide a finer adjustment of phase and that may permit further steps than those described above with respect to a frequency shifter. The ability to change the phase of the signal allows tuning for improved constructive interference of the signal as it reaches the detectors. In the example of FIG. 10, a change in phase of a carrier frequency may be used to find an improved phase at which detected signal increases. The phase shift may be continuous or in steps. For example, in FIG. 10, a sweep through 180 degrees is shown with a maximum detected signal around 20 degrees (highest constructive interference) and a minimum detected signal around 90 degrees (highest destructive interference).

In some situations, a cost of an adjustable frequency oscillator may be prohibitively expensive. As described below, a change in phase may provide similar results to a change in frequency. Further, a phase shifter may be used in addition to the frequency shifter as described above. For reference, the frequency shifter may be part of or separate from the high frequency oscillator 218. Similarly, the phase shifter may be part of or separate from the high frequency oscillator 218.

A further consideration may include not only the placement of the external antenna 110 relative to the stimulator antenna 238, but also the depth of the stimulator antenna 238. In one example, a process of adjusting a transmitted signal may comprise placing the external antenna at a location, increasing the power of the transmitted signal to a point at which the neural stimulator turns on, adjusting one or more of a frequency or a phase of the transmitted signal to determine a maximum detected signal, and adjusting one or more of the frequency or phase of the carrier signal. In one example, the system may attempt to provide a minimum power to the neural stimulator to minimize stress to non-stimulated surrounding tissues.

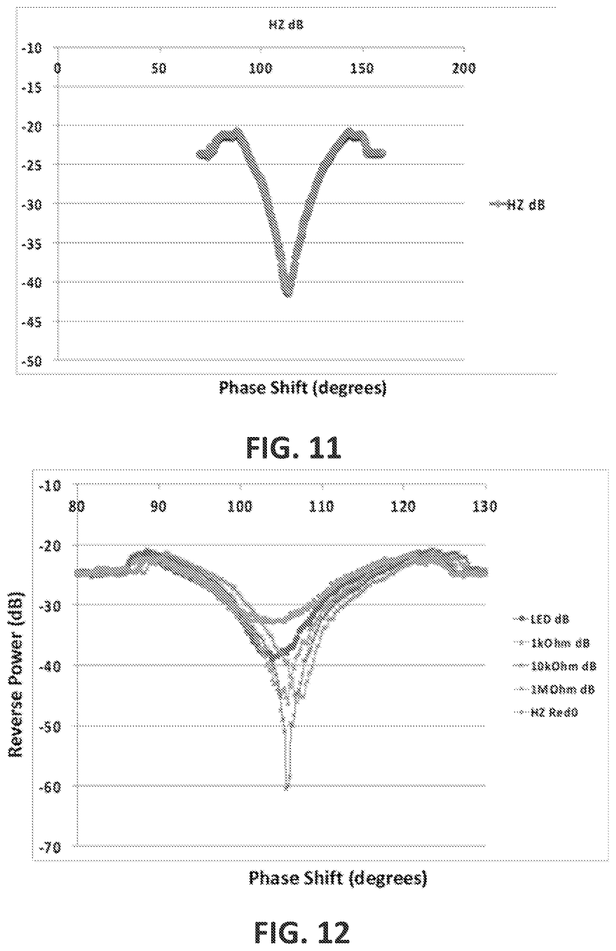

Phase shifting in some instances may also enhance power transfer to the neural stimulator. In another example, an external antenna may be relatively well matched (e.g., impedance matched) with a stimulator antenna. In the example of FIG. 11, a neural stimulator is placed in a phantom tank (a testing tank simulating the tissues of a body) and the stimulator internal circuitry is energized but not at a sufficient level to produce a backscatter signal 116, power the stimulator's circuitry, or provide an output stimulus current. The change in impedance of the neural stimulator may be a result of a change in phase. In the example of FIG. 11, there is a strong mutual coupling between the external antenna and the stimulator antenna. The neural stimulator was set in a high impedance mode (Hz). Based on the detected impedance change per phase change, the phase may be adjusted to permit a maximum power transfer and consumption by the neural stimulator, resulting in a near-perfect match in impedance (e.g., minimal to no returned RF power). F power returns). One benefit of impedance matching the external antenna to the internal antenna is a reduction in energy required to power the neural stimulator. The impedance matching may reduce a power drain on the transmitter (or separately powered external antenna), thus prolonging the use of a battery with a fixed available amount of power (e.g., decreasing how often the battery needs to be replaced or recharged).

Referring to FIG. 12, using a similar setup to that of FIG. 11, the neural stimulator is activated to drive stimulus current, and the electrode resistor load is varied, the phase shifting of the above example allows for capturing a stronger RF response to the electrode load. In this case outside of V shaped region of the backscatter power, RF reverse power variation is flat with the shift in phase. The various lines represent a sufficient power permitting the neural stimulator to turn on (e.g., LED db), 1 k Ω dB, 10 k Ω dB, 1 M Ω dB, and HZ (high impedance). There is a clear distinction between in the reverse power sweeps when the neural stimulator has different loads connected to the electrodes.

In the above graph, what is shown is the reverse power vs. phase sweep with the neural stimulator LED activated (brown squares) a 1 kΩ resistor connected to the electrodes (green diamond) 10 kΩ resistor connected to the electrodes (purple x) 1 MΩ resistor connected to the electrodes (blue x) and neural stimulator configured to high impedance (HZ) (brown circle). Note the difference between the HZ and 1 MΩ load is: in 1 MΩ case the neural stimulator is attempting to drive current, but cannot; in the HZ case, the neural stimulator is not trying to drive current, so the neural simulator power consumption is much less.

With respect to FIG. 12, the transmitting antenna may be in a blind spot (e.g., a tissue or bone or other structure prevents a clear signal reading such that one cannot tell the impedances apart. Further, if outside a certain phase, one may not be able to discern the different steps. This may be caused by long path lengths of the cables connecting the components. Moreover, if a phase shifter is placed in line with the incoming signal (e.g., using a common path for outgoing and incoming signals), the phase shifter may vary the attenuate the received signal (making it difficult to discern content in the received signal).

Using a programmable phase shifter may permit the system to find a highest difference between the transmitted and received signals, meaning more power is being transmitted to the neural stimulator. Without an ability to adjust the phase of the transmitted signal, destructive interference may prevent the ability to determine whether further adjustments of the external controller or neural stimulator are making a difference. Programmable phase shifters are known with a variety of input controls (e.g., 8-bit) and the degrees of phase adjustment (e.g., 180, 360, etc.).

With respect to FIG. 13, the use of higher RF power may put at risk the RF power amplifier on the transmitter due to excessive power being reflected by the neural stimulator antenna and received by the external antenna. The components of FIG. 13. are numbered consistently with those of the components in other figures. In FIG. 13, a circulator 1301 may be used to divert power from returning to the RF amplifier 216. The circulator 1301 may include three ports (a first port receiving an outgoing signal from the RF amplifier 216, a second port for outputting the outgoing signal via wired connection 108 and receiving an incoming signal via wired connection 108, and a third port for outputting the incoming signal to feedback subsystem 212). The third port may be terminated with a load 1302 configured to absorb reverse traveling RF power. The load 1302 may comprise real impedance (e.g., resistance via one or more resistors) or imaginary impedance (e.g., reactance via an inductor or capacitor or combination of inductors and/or capacitors) or a complex impedance (e.g., having both a resistive component and a reactive component via a combination of at least one resistor and at least one of a capacitor or inductor). A circulator may be a passive, in this example, 3-port device in which an RF signal entering any port is transmitted to the next port in rotation. With the three port circulator, a signal applied to a first port only comes out of a second port; a signal applied to the second port, only comes out of the third port; and a signal applied to the third port, only comes out of the first port. A scattering matrix (S-parameters matrix) for an ideal three port circulator is show below relating to the various inputs and related outputs (as implemented, the values may be less than one and greater than zero):

$$S = ( \begin{matrix}0 & 0 & 1 \\ 1 & 0 & 0 \\ 0 & 1 & 0\end{matrix} ) \qquad ( 4 )$$

In this example, the output of the transmitter may be connected to port 3, the external antenna may be connected to port 1, and the attenuating load may be connected to port 2, where any RF energy coming back from the external antenna may be diverted into the load connected to port 2 and be dissipated/consumed. As shown in FIG. 13, the output of the circulator 1301 is conveyed to the amplifier 226 of the feedback subsystem 212 and shunted to ground through a load 1302. The load 1302 may help dissipate dangerous spikes in received RF energy to protect the circuitry of the transmitter 106.

Data from the transmitter 106 may be acquired and transferred to an external computer for processing. This data may be acquired via one or more devices. For example, the forward and reverse RF signal can be acquired directly from the transmitter and transferred to a computer via Bluetooth, or via USB/Micro USB cable. Also, the data may be acquired via a feedback analyzer 1303 (e.g., a spectrum analyzer, an oscilloscope or other data acquisition (DAQ) system) that may be connected to the output RF detectors on an external circuit board. For example, as shown in FIG. 13, the feedback subsystem 212 is shown in dashed lines as the feedback subsystem 212 may or not be included in the transmitter 106. Where included, load 1302 may act to filter strong RF return signals from the neural stimulator to only permit some of the RF return signals to the feedback subsystem 212 (e.g., an attenuated signal, a signal with reduced DC components, etc.). Additionally or alternatively, a feedback analyzer 1303 may receive the return signal from the neural stimulator from connection 108 (e.g., before or after circulator 1301) and process it through components similar to those of the feedback subsystem 212 (e.g., an amplifier 226, a filter, 224, a demodulator 222, an analog-to-digital converter 220) and provide the output 1304 to an oscilloscope or other processor, permitting an analysis of the RF return signals. While not shown, the feedback analyzer 1303 may include other connections with the components of the transmitter 106 including, for example, a connection over which the output signal from the RF amplifier 216 is provided to the amplifier 226 of the feedback analyzer 1303.

Additionally or alternatively, the location of the circulator 1301 may be moved from inside the transmitter 106 to external to the transmitter 106 as shown by circulator 1305 being located between the transmitter 106 and the external antenna 110, such that a first port is connected to the output of RF amplifier 216 of the transmitter 106, a second port is connected to the connection 108 connected to the external antenna 110, and a third port is connected to the amplifier 226 of the feedback analyzer 1303.

Data may be sent from the neural stimulator to the external controller through a variety of techniques. For example, by changing the effective antenna length by changing connections via circuitry in the neural stimulator, the RF impedance of the neural stimulator changes. That change may be detected by the external controller.

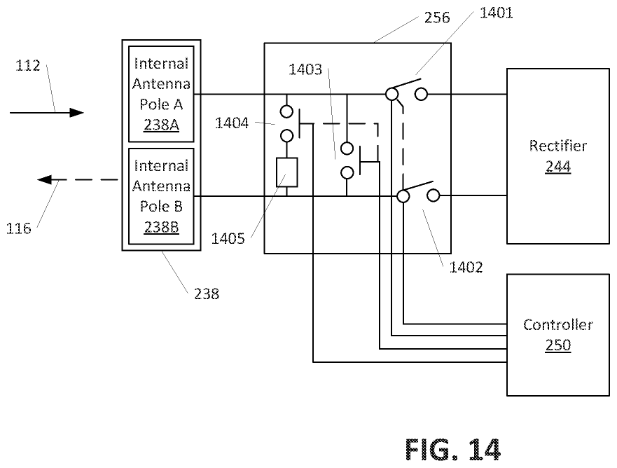

Backscatter may be modulated by the neural simulator 114 in various ways. For example, a backscatter signal 116 (e.g., a modulated RF impedance of the stimulator antenna 238) may be controlled by the circuitry of the neural stimulator 114. For example, the controller 250 of the neural stimulator 114 may control one or switches in the switching circuit 256. For instance, switching circuit 256 may comprise one or more transistors (e.g., field effect transistors or other RF-compliant transistors) that may be selectively opened and closed based on one or more control signals from the controller 250. As shown in FIG. 14, a pole A switch 1401 may be connected between stimulator antenna pole A 238A (e.g., a first terminal connected to stimulator antenna pole A 238A) and the controller 250, and may be controlled by the controller 250 to selectively open and close switching circuit 256 to change the RF impedance of stimulator antenna 238. Additionally or alternatively, a pole B switch 1402 may be connected between stimulator antenna pole B 238B (e.g., a first terminal connected to the stimulator antenna pole B 238B) and controller 250, and may be controlled by the controller 250 to selectively open and close switching circuit 256 to change the RF impedance of stimulator antenna 238. For example, the pole A switch 1401 may be controlled by the same or different control signal from controller 250 that controls the pole B switch 1402 (different control signals shown by different control lines, the same control signal shown by the broken line connecting switches 1401 and 1402). Additionally or alternatively, a shorting switch 1403 may be have one terminal connect to the stimulator antenna pole A 238A and another terminal connected to stimulator antenna pole B 238B and may be controlled by the controller 250 to selectively open and close to change the impedance of the stimulator antenna 238. Additionally or alternatively, a load energizing switch 1404 may connect a load 1405 between the stimulator antenna pole A 238A and the stimulator antenna pole B 238B. The load 1405 may comprise a resistor, a diode (e.g., a light emitting diode (LED)), and/or another device or devices. Where comprising a light emitting diode and when the load energizing switch 1404 is operated to connect the light emitting diode between the stimulator antenna pole A 238A and the stimulator antenna pole B 238B, light from the diode 1405 (where diode 1405 is an LED) may permit one to visually determine that power is being received by the neural stimulator 114. The load energizing switch 1404 may be controlled by the controller 250 to selectively open and close to change the impedance of the stimulator antenna 238. The load energizing switch 1404 may be controlled separately from the shorting switch 1403 or both may be controlled via a common control signal from the controller 250 (shown by the dashed line connecting load energizing switch 1404 to switch 1403). The switches 1401-1404 may be controlled to selective connect and/or selectively disconnect their conduction paths between their input and output terminals.

One or more of the pole A switch 1401, the pole B switch 1402, the shorting switch 1403, or load energizing switch 1404 may be operated independently. Additionally or alternatively, they may be operated in conjunction with one or more of the other switches. The parameter-setting attributes from controller may comprise instructions to change an operation mode of the neural stimulator 114 between, e.g., a location-determination mode, an impedance sensing mode, a testing mode, and/or a stimulation mode. For various combinations of activations of the switches are shown in Table 2:

TABLE 2

| | Pole A | Pole B | Shorting | Load | |
|---|---|---|---|---|---|
| | Switch | Switch | Switch | Energizing | |
| State | 1401 | 1402 | 1403 | Switch 1404 | Result |
| 1 | On | On | Off | Off | Power |
| | | | | | Transmitted |
| | | | | | to rectifier 244 |
| 2 | Off | On | Off | Off | Open circuit |
| 3 | On | Off | Off | Off | Open circuit |
| 4 | Off | Off | Off | Off | Open circuit |
| 5 | Don't | Don't | On | Off | Short circuit |
| | Care | Care | | | |
| 6 | On | On | Off | On | LED powered |

| | | | | |
|---|---|---|---|---|
| 7 | One or more Off | Off | On | LED powered |

Selected states are shown in Table 2. Other states are possible but not shown for simplicity. Further, it is appreciated that removing one or more of switches 1401-1404 or adding additional switches may affect the number of possible states. Also, for reference, the switches are described as “On” meaning “conducting” and “Off” as “not conducting”. It is appreciated that these definitions may be switched as needed and relevant to the types of transistors used (e.g., p-type field effect transistors, n-type field effect transistors, etc.).

In state 1, both the pole A switch 1401 and the pole B switch 1402 are conducting received RF energy to rectifier 244. In states 2, 3, and 4, at least one of the pole A switch 1401 and the pole B switch 1402 are not conducting, resulting in an open circuit and no power being transferred to rectifier 244. In state 5, the shorting switch 1403 is conducting and creates a short circuit between the poles A and B of the stimulator antenna 238. In state 6, the load 1405 is connected in parallel with rectifier 244. While not shown, one or more additional diodes (including but not limited to conventional diodes, LEDs, Zener diodes, and the like) may be placed in series with the rectifier 244 to provide an indication of whether power is being transmitted to the rectifier 244. In state 7, the load 1405 is receiving all power from the stimulator antenna 238 and no power is being received by the rectifier 244. Based on the configuration of the circuitry and options set in the controller 250, state 7 may be an unrecoverable state as, to switch out of state 7, power may need to be received at the rectifier 244 and then provided to the controller 250. As an unrecoverable error state, state 7 may protect the patient by preventing further operation a defective neural stimulator. Alternatively, state 7 may only be a temporary state to temporarily reduce the power received by the rectifier 244. For example, the load energizing switch 1404 (and/or the shorting switch 1403) may be open (non-conducting) when receiving no control signal from the controller 250. When a power level received by the rectifier 244 is above a threshold, the controller 250 may temporarily energize one of the load energizing switch 1404 or the shorting switch 1403 to reduce the power received by rectifier 244. Through the use of a timing circuit in controller 250 (e.g., a capacitor and load) (not shown), one or more of the load energizing switch 1404 or the shorting switch 1403 may be powered for a time T to short the poles of the stimulator antenna 238 or energize load 1405. Once the power in the timing circuit is below the threshold voltage for the one of the load energizing switch 1404 or the shorting switch 1403, relevant switch no longer conducts and the received RF signal is again provided to rectifier 244.

Further, with respect to state 6, when the two antenna arms (antenna poles A-B 238A-238B) are connected together via the shorting switch 1403 (e.g., also referred to as backscatter FETs), the resulting estimated effective resistance may be approximately 20 ohms for example. A backscatter signal 116 produced by toggling the backscatter FETs state so that the antenna poles A-B 238A-238B may be switched between shorted together and open conditions at the feed port. When the antenna arms are shorted together the stimulator antenna becomes one long wire, when open, the antenna arms become a dipole antenna with the energy feeding into the rectifier 244.

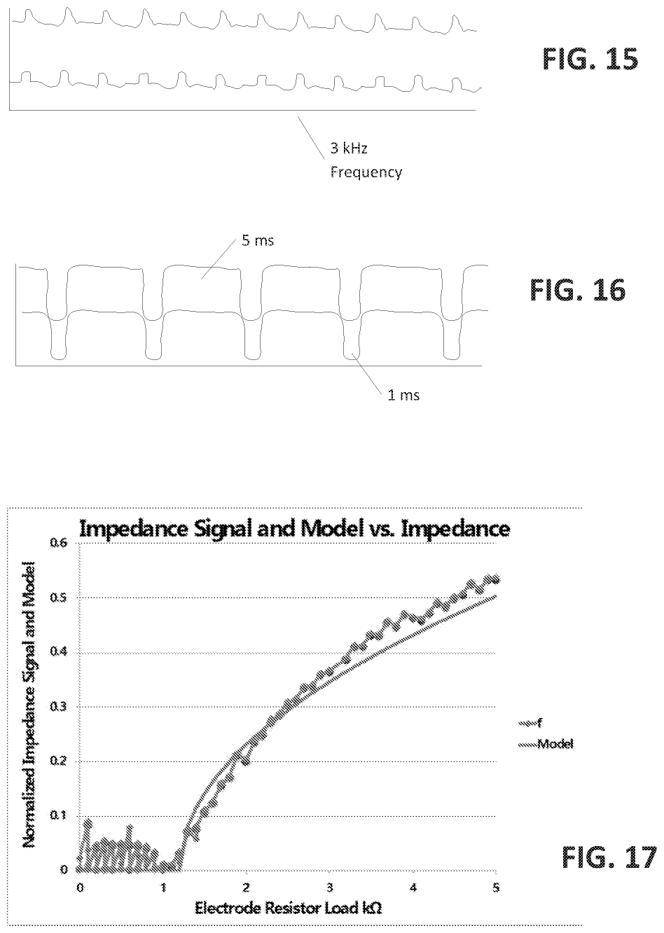

FIGS. 15 and 16 are two examples are of signal that results from changing the RF antenna via changing of the neural stimulator 114's settings. In FIG. 15, a timing circuit for an LED is set for 1000 ms on where the only load on the neural stimulator 114 is the stimulator LED (e.g., load 1405). To test outside of a patient, a neural stimulator, may be positioned relative to an external antenna (e.g., external antenna 110). In this example, the neural stimulator and external antenna may be spaced by approximately 50 cm and the external antenna connected to a spectrum analyzer (e.g., a USB-SA44B spectrum analyzer manufactured by Signal Hound of Battle Ground, Wash.). The data may then be processed and plotted, e.g., in LabVIEW (National Instruments of Austin, Tex.), with a graphical user interface of LabVIEW.

The transmitter 106 may be set to a high pulse rate in order to sense the timing of the neural stimulator 114 switching of connections. This high rate may allow for the internal circuitry of the neural stimulator 114 to use a leading edge of the received RF signal to trigger the next current pulse so that the current pulses are sequentially continuous—one right after the other. The spectrum analyzer may also be set to zero-span at the transmitter 106's carrier center frequency. The resulting data may be collected and processed—e.g., with a smoothing of the determined curve performed with a running average of 100 samples at that a pulse rate of 3 kHz for the transmitter 106.

In FIG. 16, the neural stimulator 114's electrode settings are set to 12 mA with 1 ms of stimulus pulse and 5 ms of active balancing time. The timing of the connections may be set for optimal detection. In this example, there is a 5 ms and 1 ms signal change pattern. Also, the signal from a neural stimulator 114 may be different based on which electrodes are energized—as a resulting signal may vary based on which electrode and how many electrodes are active as well as the polarity of each electrode.

The current settings as shown in FIGS. 15 and 16 may be too powerful for use in a patient at these power settings and pulse rate but at least a lower power signal may be used. These examples of settings and sensing may be used to verify that the neural stimulator is functioning correctly before insertion into a patient or after insertion while energizing the electrodes to levels or frequencies such that the stimulation is not painful to the patient.

With respect to FIG. 17, observation of the RF signal may be used to sense an impedance of the tissue surrounding the neural stimulator electrodes via the RF response. In the example of FIG. 17, the transmitter 106 is connected to the external antenna 110. The external antenna 110 radiates the RF signal into the air. The external antenna 110 may be switched to act as a receiving antenna or a second antenna may be used as the receiving antenna. In the example where a second antenna is used, the second antenna may be located parallel to the external antenna 110. This two-antenna system may permit some of the RF energy and signal to escape and to radiate into the room surrounding the setup. A third antenna may be used as a receiving antenna may be placed approximately 50 cm away from the first two antennas. The third antenna may be connected to a spectrum analyzer and the third antenna moved until a strong signal from the first antenna is received. The signal data may be sampled via a spectrum analyzer and the processed on a computer (e.g., using LabVIEW).

In order to extract the impedance information, three neural stimulator settings may be set (from parameter-setting attributes from the transmitter) to produce the signals useable to extract impedance information. After a system calibration where the power needed to activate and drive the neural stimulator is known, the impedance signal may be defined with the RF response to three configurations:
Signal 1: RF Response to a programmed LED current (LED) (at which point the stimulator internal circuitry beings operating).
Signal 2: RF Response to programmed High Impedance (HZ) where all the electrodes are inactive.
Signal 3: RF Response to programmed stimulus current (Stim)

Signals 1 and 2 may be used to define the signal strength and signal-to-noise ratio (SNR), and for normalization of the stimulus current signal. Signal 3 may be recorded as the simulated tissue load is swept from a zero load through a range of physiologically relevant loads for neural stimulator applications (e.g., from zero through a resistive value over 5 k Ω or larger).

The three signals—Signals 1-3—are based on a steady state voltage on a rectifier and a capacitor bank. That voltage may change based on the amount of current that is being driven through the circuitry of the neural stimulator. The neural stimulator rectifier voltage may swing between about 4 to about 10 V while the circuitry is active.

The RF power settings are recorded, and then held constant during these measurements. The neural stimulator may be programmed for three different configurations and the radiated signal response may be picked up by the antenna in air and directed to the spectrum analyzer. The data is then averaged and stored. These measurements may then be processed so that the impedance at the electrodes may be extracted.

In order to extract the impedance value from the signal a data set of the three signals should be made for a range of electrode load values. A sweep of the electrode load, while everything else is held constant, may provide response data for the three signals from which an impedance model may be derived.

Normalizing the impedance signal vs. electrode load sweep results in the plot of FIG. 17. A mathematical model may be approximated to fit to the data. The data of the received signal is plotted as circles connected by lines and the curve of approximated model is shown as a solid line. The electrode resistive load is shown on the X-axis in k Ωs and the normalized impedance and model are shown on the Y-axis.

In this example, the normalization of the impedance RF signal for the measurements may be determined as shown below in equation (5):

f=(LED−Stim)/(LED−HZ) (5)

The mathematical model approximation may be represented as equation (6) below:

Model=sqrt((Z−A)/B) (6)

In equation (6), A is the impedance value at which the RF signal begins to change (rise above the noise floor) and may be distinguished from the noise floor (in this example, approximately ˜1 kΩ), and B is a scaling factor dependent on relative signal strengths of the measurement test setup, and Z is the estimated electrode-tissue impedance.

Using the model's equation, the impedance may be extracted from the normalized data taken from the setup, in this example, the inverse of the model function may be used to solve for the impedance (Z) as a function of the normalized signal.

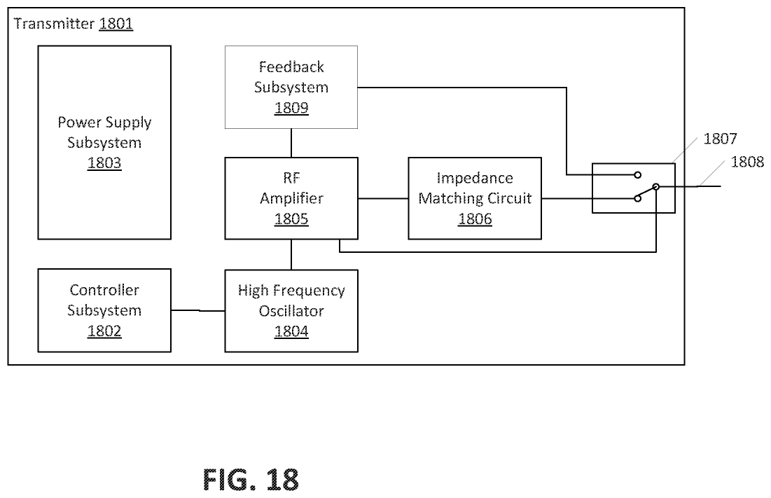

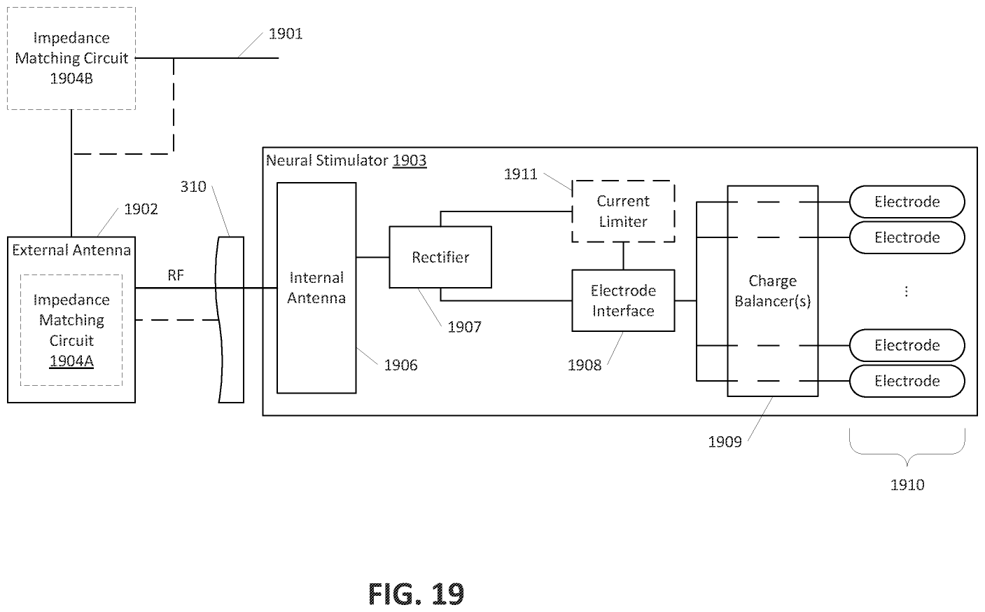

Using the equation (6), the impedance of a neural stimulator in a patient may be estimated. For example, an impedance matching circuit as shown in FIG. 18 may be used. In FIG. 18, a transmitter 1801 includes a controller subsystem 1802 and a power supply subsystem 1803, powering the transmitter 1801. Signals from the controller subsystem 1802 are sent to a high frequency oscillator 1804 where the signals modulate a carrier signal. The composite signal is sent to RF amplifier 1805. The RF amplifier 1805 amplifies the composite signal. The composite signal is sent, via impedance matching circuit 1806 to switch 1807 for forwarding to connection 1808 to a neural stimulator (not shown). The impedance matching circuit 1806 may be adjusted to more closely match the impedance of an external antenna to the tissue impedance determined via equation (6). Alternatively or additionally, an impedance matching circuit in a connection to an external antenna or in the external antenna may be used as shown in FIG. 19. A composite signal is received over connection 1901 and received by an external antenna 1902. The composite signal is radiated to the neural stimulator 1903 and received by an internal antenna 1906 of the neural stimulator 1903. The received signal is rectified by rectifier 1907. The output of the rectifier 1908 is received by one or more charge balancers 1909 and output to electrodes 1910 as stimulation currents. FIG. 19 also includes one or more impedance matching circuits 1904A and 1904B. The impedance matching circuit 1904A is located in the external antenna 1902 and, for example, no impedance matching circuit located in connection 1901. Alternatively or additionally, the impedance matching circuit 1904B may be located in connection 1901 between the transmitter and the external antenna 1902. An impedance matching circuit (one or more of 1904A or 1904B) may be adjusted to more closely match the impedance of the external antenna 110 to the electrode-tissue impedance determined via equation (6).

Another process for determining the impedance may comprise observing the voltage across the neural stimulator rectifier and its resistance-capacitance (RC) time constant. For example, the RF response that corresponds to the drop of rectifier voltage just after the neural stimulator circuitry activation (where current is subsequently driven through an electrode load). The time constant (the time RC constant, τ or Tau) for the time the rectifier voltage to drain may be used to extract the resistance R, from the RC constant, and estimate the resulting impedance surrounding the neural stimulator.

Based on the estimate of the electrode-tissue impedance surrounding the neural stimulator electrodes, the external antenna may be adjusted based on the electrode-tissue impedance, thereby improving the power transferred to the neural stimulator electrodes. With improved impedance-matching, a lower power level may be used to power the neural stimulator at a desired power level. This is in comparison to a poorly matched impedance where a greater power level would be required to power the neural stimulator at the desired power level.

A number of implementations have been described. Nevertheless, it will be understood that various modifications may be made. Accordingly, other implementations are within the scope of the following claims.

What is claimed is:

1. An apparatus comprising:
an antenna comprising a first pole and a second pole;
a switching circuit configured to receive a first pole signal from the first pole and configured to receive a second pole signal from the second pole, wherein the switching circuit is configured to output switched signals;
a rectifier configured to receive switched signals from the switching circuit;
a plurality of electrodes; and
a controller configured to receive power from the rectifier, configured to selectively power the electrodes, and configured to output a control signal to the switching circuit,
wherein the switching circuit, based on the control signal, modifies one or more of the first pole signal or the second pole signal.

2. The apparatus of claim 1, wherein the controller is further configured to:
receive instructions, from an external controller, to modify the control signal, and output, based on the instructions, the control signal.

3. The apparatus of claim 1, wherein the switching circuit comprises:
a first switch comprising an input configured to receive the first pole signal and configured to output the first pole signal as one of the switched signals,
wherein the first switch, based on the control signal, prevents the first pole signal from being output as one of the switched signals.

4. The apparatus of claim 3, wherein the controller is configured to output a second control signal, and wherein the switching circuit further comprises:
a second switch comprising an input configured to receive the second pole signal and configured to output the second pole signal as another of the switched signals,
wherein the second switch, based on the second control signal, prevents the second pole signal from being output as the another of the switched signals.

5. The apparatus of claim 1, wherein the switching circuit comprises:
a first switch comprising an input configured to receive the first pole signal and configured to output the first pole signal as one of the switched signals; and
a second switch comprising an input configured to receive the second pole signal and configured to output the second pole signal as another of the switched signals,
wherein the first switch, based on the control signal, prevents the first pole signal from being output as one of the switched signals, and
wherein the second switch, based on the control signal, prevents the first pole signal from being output as one of the switched signals.

6. The apparatus of claim 1, wherein the switching circuit comprises:
a first switch comprising a first terminal connected to the first pole and a second terminal connected to the second pole,
wherein the first switch, based on the control signal, shorts the first pole and the second pole.

7. The apparatus of claim 1, wherein the switching circuit comprises:
a first switch comprising a first terminal and a second terminal, wherein the first terminal is connected to the first pole; and
a load connected between the second terminal and the second pole,
wherein the first switch, based on the control signal, connects the load to the first pole.

8. The apparatus of claim 7,
wherein the load is a diode.

9. The apparatus of claim 7, wherein the load is a diode, and further comprising:
a second switch comprising a third terminal connected to the first pole and a fourth terminal connected to the second pole,
wherein the second switch, based on the control signal, shorts the first pole and the second pole.

10. The apparatus of claim 1,
wherein the switching circuit modifies an impedance of the apparatus.

11. A method comprising:
receiving, at an antenna, a radio frequency signal, wherein the antenna comprises a first pole and second pole, and wherein the antenna comprises a first impedance;
receiving, via an input of a switching circuit from the antenna, the radio frequency signal;
selectively outputting, via an output of the switching circuit and based on a control signal from a controller, a switched radio frequency signal; and
receiving, at a rectifier, the switched radio frequency signal,
wherein the selectively outputting interrupts, based on the control signal, a conduction path between the input of the switching circuit and the output of the switching circuit,
wherein the controlling operation modifies the antenna to comprise a second impedance, and
wherein the second impedance is different from the first impedance.

12. The method of claim 11, wherein the selectively outputting comprises:
receiving, via a control signal line, the control signal; and
modifying, based on the control signal, a conduction between a first terminal of a switch of the switching circuit and a second terminal of the switch of the switching circuit,
wherein the modifying the conduction of the switch creates on open circuit between the first terminal and the second terminal.

13. The method of claim 12, wherein the selectively outputting further comprises:
modifying, based on the control signal, a conduction between a third terminal of a second switch of the switching circuit and a fourth terminal of the second switch of the switching circuit,
wherein the modifying the conduction of the switch creates on open circuit between the third terminal and the fourth terminal.

14. The method of claim 12, wherein the selectively outputting further comprises:
modifying, based on a second control signal, a conduction between a third terminal of a second switch of the switching circuit and a fourth terminal of the second switch of the switching circuit,
wherein the modifying the conduction of the switch creates on open circuit between the third terminal and the fourth terminal.

15. The method of claim 11, wherein the selectively outputting further comprises:
modifying, based on the control signal, a conduction between a first terminal of a first switch of the switching circuit and a second terminal of the second switch of the switching circuit,
wherein the first terminal is connected to the first pole of the antenna,
wherein the second terminal is connected to the second pole of the antenna, and
wherein modifying the conduction comprises creating a short circuit between the first pole and the second pole of the antenna.

16. A method comprising:
determining a first impedance at which a neural stimulator begins to respond to an input radio frequency signal from an external antenna and a normalized impedance radio frequency signal rises above a background noise floor;
determining a scaling factor dependent on relative signal strengths of the measurement test setup;
determining, based on the first impedance value, and the scaling factor, an estimated electrode-tissue impedance of the neural stimulator; and
outputting the estimated electrode-tissue impedance.

17. The method of claim 16, wherein the determining the estimated electrode-impedance of the neural stimulator is based on a model of normalized impedance RF signal, where:
Model=sqrt((Z−A)/B)
wherein Z is the estimated electrode-tissue impedance for the neural stimulator,
wherein A is the impedance value at which the normalized impedance RF signal rises above a background noise floor, and
wherein B is a scaling factor dependent on relative signal strengths of the measurement test setup.

18. The method according to claim 16, wherein based on the estimated electrode-tissue impedance of the neural stimulator, an impedance of the external antenna is adjusted based on an impedance matching circuit of the external antenna.

19. The method according to claim 15,
wherein, based on the estimated electrode-tissue impedance of the neural stimulator, an impedance of the external antenna is adjusted by means of an impedance matching circuit of the transmitter.

20. A method comprising:
receiving a radio frequency signal at an antenna of a neural stimulator;
determining a voltage across a rectifier of the neural stimulator;
determining a time, based on phases of the radio frequency signal, of a voltage drop across the rectifier;
determining, based on voltage across the rectifier and the time, a resistance-capacitance time constant of the neural stimulator;
determining, based on the resistance-capacitance time constant, an electrode-tissue impedance of the neural stimulator; and
outputting the estimated electrode-tissue impedance.

21. The method according to claim 20, wherein based on the electrode-tissue impedance of the neural stimulator, an impedance of the external antenna is adjusted based on an impedance matching circuit of the external antenna.

22. The method according to claim 20,
wherein, based on the estimated electrode-tissue impedance of the neural stimulator, an impedance of the external antenna is adjusted by means of an impedance matching circuit of the transmitter.
